(19) United States
(12) Patent Application Publication
(10) Pub. No.: US 2026/0283826 A1
(43) Pub. Date: Sep. 24, 2026

(54) Dynamic Hip Orthosis Device

(71) Applicant: Icarus Medical, LLC, Charlottesville, VA (US)

(72) Inventors: David T. Johnson, Charlottesville, VA (US); Evan Eckersley, Charlottesville, VA (US); Philip Miller, Charlottesville, VA (US)

(21) Appl. No.: 19/687,008

(22) Filed: May 25, 2026

Related U.S. Application Data
(63) Continuation of application No. 18/669,479, filed on May 20, 2024, now Pat. No. 12,636,176.
(60) Provisional application No. 63/467,464, filed on May 18, 2023.
(60) Provisional application No. 63/468,763, filed on May 24, 2023.

(51) Int. Cl. A61F 5/01

(52) U.S. Cl. CPC A61F 5/0104; A61F 5/0193

(57) ABSTRACT

A hip orthosis of the hip joint for treatment of hip osteoarthritis or post-operative recovery. The hip orthosis includes embodiments of tensioning elements, compression elements, and elements to store and release energy across, in, or around, the hip joint. The device allows for a custom adjustment of the functionality by the wearer using an adjustment component.

### CROSS-REFERENCE TO RELATED APPLICATIONS

The present application relies on the disclosures of and claims priority to and the benefit of the filing date of U.S. patent application Ser. No. 18/669,479, filed May 20, 2024, which relies on the disclosures of and claims priority to and the benefit of the filing dates of the following U.S. Provisional Patent Applications:
U.S. Appl. No. 63/467,464, Dynamic Hip Orthosis Device, filed May 18, 2023; and
U.S. Appl. No. 63/468,763, Dynamic Hip Orthosis Device, filed May 24, 2023.

The disclosures of those applications are hereby incorporated by reference herein in their entireties.

### BACKGROUND

Existing hip joint orthoses are subject to several clinical indications and related biomechanical deficiencies, including those related to the cartilage and those related to the muscles and ligaments surrounding the joint, which is improved upon by the embodiments described herein.

Trendelenburg gait is an abnormality in the walking gait. If the gluteus medius muscle or its attachment ligaments are damaged, the muscle may not have enough strength to support the pelvis in a level position when the opposing leg is in the swing phase of the walking gait. This causes the side of the pelvis opposite of the weakened gluteus medius muscle to drop. People afflicted with Trendelenburg gait typically arch their torso towards the side of the damaged muscle to bring their center of gravity over the afflicted leg. This results in an uneven, lurching gait and can contribute to comorbidities in the spine and back.

Osteoarthritis (OA) of the hip joint is an affliction that is estimated to affect up to 20% of the worldwide population who are older than 55. It is a degenerative disease of the cartilage that leads to diminished spacing between the femoral head and the pelvic socket that can be diagnosed radiographically. As reported in “The prevalence of hip osteoarthritis: a systematic review and meta-analysis” by Fan, Z., Yan, L., Liu, H. et al. published in Arthritis Res Ther 25, 51 (2023), hip joint OA affects men and women equally. It is estimated that 1-2% of the worldwide GDP is spent on treatments for OA.

Treatment for hip OA include NSAIDs, steroid shots, and surgery.

Hip osteoarthritis is a progressive degenerative disease of the hip joint in which articular cartilage wears down, the joint space narrows, and loading across the femoral head and acetabulum becomes increasingly painful during weight-bearing activities. As degeneration advances, patients commonly experience groin or lateral hip pain, stiffness after rest, reduced range of motion, and progressive difficulty with walking, climbing stairs, rising from a chair, and other daily tasks. Because the hip is a major load-bearing joint, symptoms are often aggravated by standing and ambulation, and many patients develop compensatory movement patterns that further reduce function and quality of life.

Recovery after hip surgery, including arthroplasty and other joint-preserving or reconstructive procedures, commonly requires a period of protected mobility while tissues heal and patients gradually restore strength, balance, and confidence with weight bearing. Early rehabilitation typically focuses on controlling pain and swelling, protecting the surgical site, and re-establishing safe gait mechanics; however, many patients continue to experience discomfort during stance, asymmetrical loading, weakness, and guarded movement patterns for weeks or months after surgery. In this setting, an orthotic intervention that can reduce joint loading while allowing functional mobility may help support rehabilitation, improve tolerance for walking and exercise, and facilitate a smoother return to activity.]

The hip orthosis in aspects described herein address current treatment gaps around these conditions by applying a controlled unloading or distraction force across the hip joint, thereby reducing compressive contact at the femoroacetabular interface during standing and ambulation. By decreasing peak joint loading and redistributing forces away from diseased or recovering articular surfaces, such a device may reduce pain, improve tolerance for movement, and help preserve or restore functional range of motion. In some embodiments, the distraction force may be adjustable by the wearer to accommodate different activities or stages of recovery, allowing greater unloading during painful or demanding tasks and lower levels of assistance during routine use. In other aspects, an abduction assist function may provide similar unloading benefits to the joint by assisting the surrounding muscles, and may be incorporated with or independently from such joint distraction mechanisms. This capability may be particularly beneficial for patients with hip osteoarthritis who are not ready for surgery, as well as for post-operative patients who would benefit from partial unloading during rehabilitation.

### DESCRIPTION OF RELATED ART

Existing passive hip orthoses are limited in that they generally function only as range-of-motion control devices and do not provide a dynamic or unloading force to the hip joint.

Conventional designs typically rely on rigid hinges, shells, or straps to restrict flexion, extension, abduction, adduction, or rotation, which may be useful for protecting tissues or preventing undesirable motion but does little to reduce compressive forces across the joint surfaces. As a result, such devices may stabilize the hip without meaningfully unloading the femoral head from the acetabulum, and therefore may offer limited benefit for conditions in which pain is driven by joint contact forces, such as hip osteoarthritis or painful post-operative weight bearing.

Traditional compression and Range of Motion (ROM) hip orthoses are not effective at treating hip osteoarthritis, post-operative recovery, or Trendelenburg gait. Compression hip orthoses are garments made of an elastic cloth (e.g. Lycra) which are used to compress the hip and thigh muscles. They are useful for preventing hip displacement to some degree, but face the same or greater limitations than ROM hip orthoses in terms of efficacy.

ROM hip orthoses have a waist portion and thigh portion connected by a rigid hinge or hinges. Stops are employed to limit the rotation of the hinges and hence the range of motion of the leg and hip. ROM hip orthoses are passive devices that are used to limit the flexion/extension or abduction/adduction motion of the hip.

What is needed is a hip orthosis capable not merely of limiting motion, but of generating clinically and anatomically relevant dynamic forces including actively generating a distraction or unloading force, abduction assistance by supplementing the gluteus medius or other muscle groups, or that can relieve joint stress while preserving functional mobility.

### SUMMARY OF THE EMBODIMENTS

A cabling system, as used herein, includes flexible yet substantially inextensible or inextensible materials such as wire cable, braided cord, string, lace, chain, rope, straps, banding, ribbon, and the like.

A torso component, as used herein, is the part of a hip orthosis that is predominantly located above the hip joint. A waist component is a type of torso component that is predominantly located about the waist. A chest component is a type of torse component that is predominantly located about the chest.

A thigh component, as used herein, is the part of a hip orthosis that is predominantly located below the hip joint.

The effected leg, as used herein, is the leg with the damaged gluteus medius muscle and/or OA and upon which a leg portion of the orthosis is attached. The opposite leg, as used herein, is the leg that does not have the damaged gluteus medius muscle or OA. For the sake of clarity, in the examples below, it is assumed that only one of the gluteus medius muscles is damaged and/or has OA and that person is only wearing the hip orthosis on one side. This does not preclude situations where a person would wear the hip orthosis disclosed herein on both sides of their body, as there are individuals with damaged gluteus medius muscles and/or OA in both legs.

The term anchor mount refers to the physical element (or elements) of the connection component that is attached to the torso or leg component. The term anchor point refers to the location of the anchor mount on the torso or leg component, by way of example.

### BRIEF DESCRIPTION OF THE DRAWINGS

The accompanying drawings illustrate certain aspects of embodiments of the present invention and should not be used to limit the invention. Together with the written description the drawings explain certain principles of the invention.

FIG. 1 is an illustration of the bones in a human hip joint.

FIG. 2 is an illustration of the three orthogonal planes of the human body.

FIG. 3 shows a schematic illustration of a compression type hip brace.

FIG. 4 shows a schematic illustration of a hip brace with a tensioning mechanism that applies a force across the hip joint.

FIG. 5 shows a schematic illustration of a hip brace with an adjustable attachment mechanism for the tensioning element.

FIG. 6 shows a schematic illustration of a hip brace with a cam/lever mechanism to engage and disengage the tension force across the hip joint.

FIG. 7 shows a schematic illustration of a hip brace with a mechanism to increase the force applied across the hip joint compared to the force applied to the tensioning tab.

FIG. 8 shows a schematic illustration of a dynamic hip brace wherein the magnitude and direction of the force vector across the hip joint varies with the position of the wearer's leg.

FIGS. 9a and 9b show an illustration of a hip orthosis suitable for controlling hip dislocation.

FIG. 10 shows an illustration of the hip orthosis of FIG. 9b with a ratchet and pawl dial.

FIGS. 11a and 11b show an illustration of an embodiment of a hip orthosis suitable for controlling hip dislocation that uses conformable fingers to control the forces across the hip joint.

FIG. 12 shows an illustration of a hip orthosis with a passive, secondary tension element.

FIG. 13 shows an illustration of a hip orthosis with an active, secondary tension element.

FIG. 14 shows an illustration of a distraction hip orthosis.

FIG. 15 shows an illustration of an unloading hip orthosis.

FIG. 16 shows an orthogonal view of a post hip surgery hip orthotic.

FIG. 17 is an enlargement of the hinge component of FIG. 16.

FIG. 18 is a front view of a hip orthotic with two independent energy storage elements to apply forces along two different axes of the hip joint.

FIG. 19a is an orthogonal view of a hemispherical hinge.

FIG. 19b is a sectional view of the hinge of FIG. 19a.

FIG. 19 c is an orthogonal view of the hinge of FIG. 19 a when rotated about the z axis.

FIG. 19 d is an orthogonal view of the hinge of FIG. 19 a when rotated about the x axis.

FIG. 20a is an orthogonal view of a hemispherical hinge with a pin slot.

FIG. 20b is a sectional view of the hinge of FIG. 20a.

FIG. 21 is an orthogonal view of a multi-axis hinge with a gait control mechanism.

FIG. 22. is a top down view of the gait resulting from the hinge of FIG. 21.

FIG. 23 is an illustration of the walking gait cycle.

FIG. 24 is an illustration of the pelvis drop during Trendelenburg gait.

FIG. 25 is a front view of a hip orthosis, according to embodiments of the present invention.

FIG. 26a and FIG. 26b are front views of another embodiment of the hip orthosis that employ a chest component.

FIG. 27 is an illustration of a unitary version of an embodiment of the present invention.

FIG. 28 is a depiction of how the distance between anchor points on the waist component and the thigh component changes during a walking gait, according to embodiments of the present invention.

FIG. 29a and FIG. 29b are illustrations of the mechanisms that can be used to change the tensile force applied by the hip brace depending on the flexion/extension position of the effected leg, according to embodiments of the present invention.

FIG. 30a, FIG. 30b, and FIG. 30c are illustrations of another aspect to change the tensile force applied by the hip brace depending on the flexion/extension position of the effected leg, according to embodiments of the present invention.

FIG. 31 is an illustration of a ROM mechanism used in conjunction with the abduction assist, according to embodiments of the present invention.

FIG. 32 is an illustration of a variation of the orthosis that can assist with adduction, according to embodiments of the present invention.

FIGS. 33a and 33b are illustrations of the femoral head and pelvic socket.

FIG. 34 is an illustration of a distraction hip orthosis.

FIG. 35 is an illustration of another embodiment of a distraction hip orthosis.

FIG. 36a is an illustration of yet another embodiment of a distraction hip orthosis.

FIG. 36b is an enlarged view of the distraction mechanism shown in FIG. 36a.

FIG. 37 is an illustration of yet another embodiment of a distraction hip orthotis.

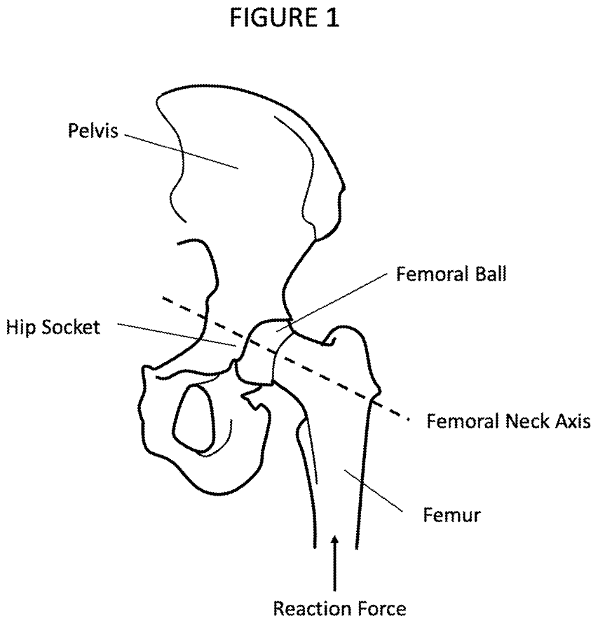

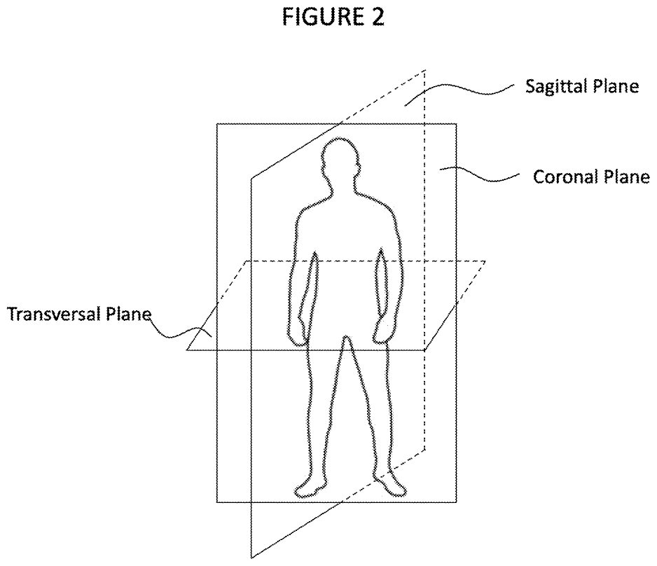

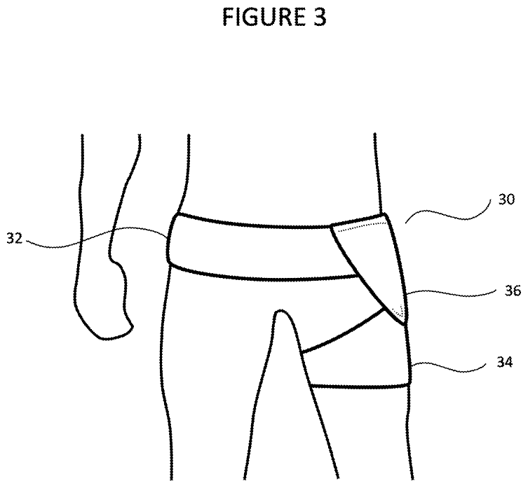

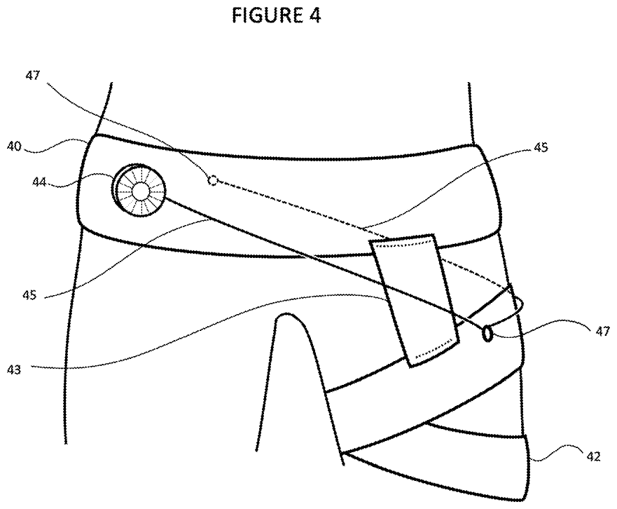

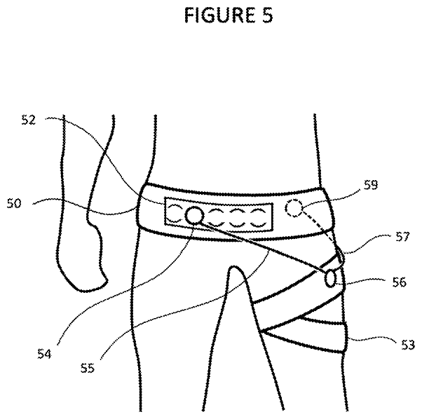

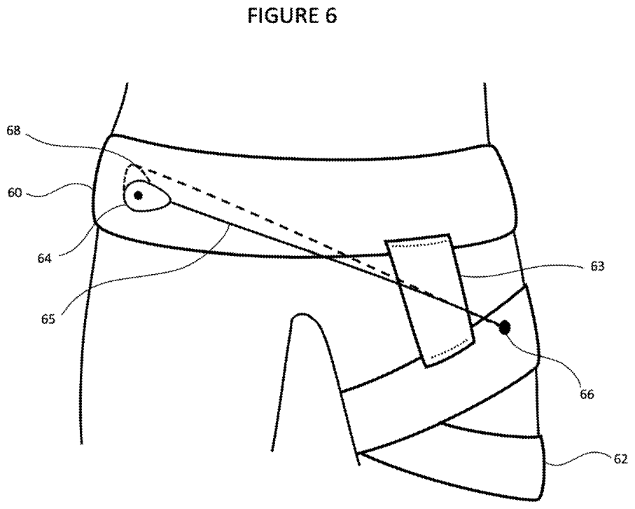

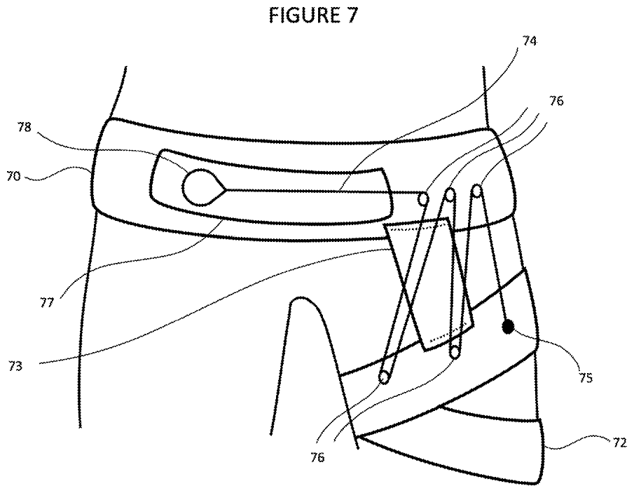

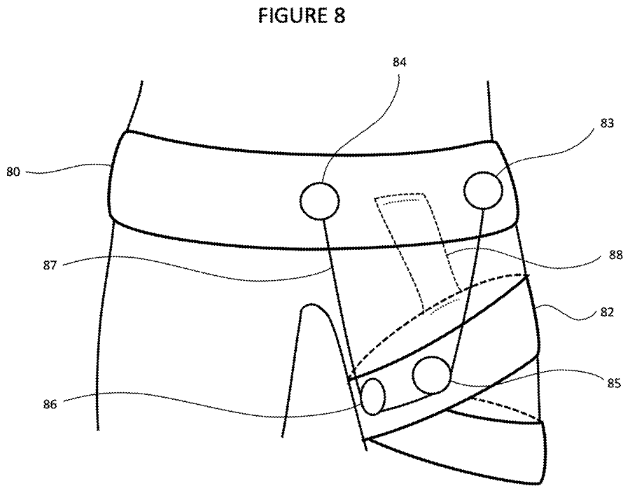

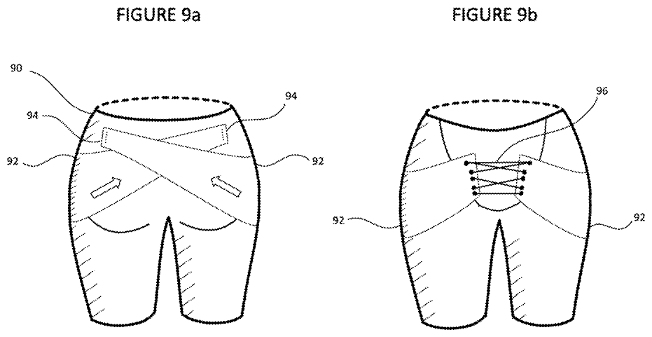

### DETAILED DESCRIPTION OF THE EMBODIMENTS

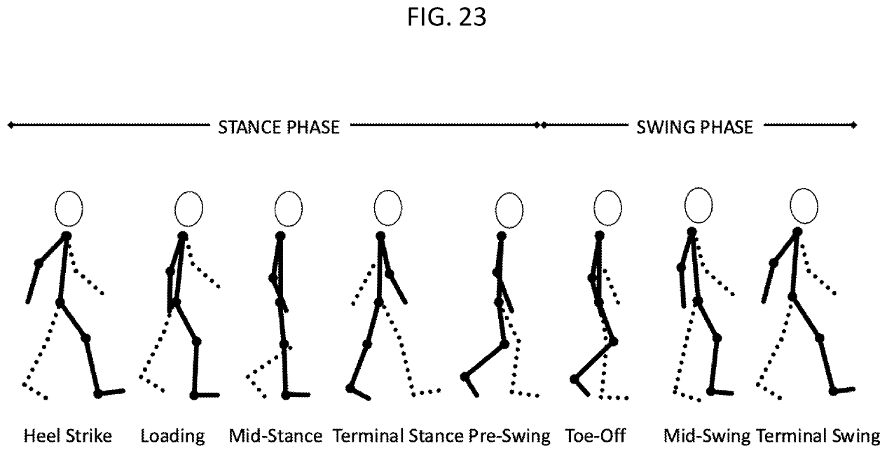

FIG. 23 shows the human gait cycle as would be understood by one of ordinary skill in the art, which is composed of a stance phase and a swing phase. In the stance phase, the figure in the illustration has all their weight supported by their right leg. In the swing phase, the weight is shifted to the left leg and the right leg is lifted off the ground.

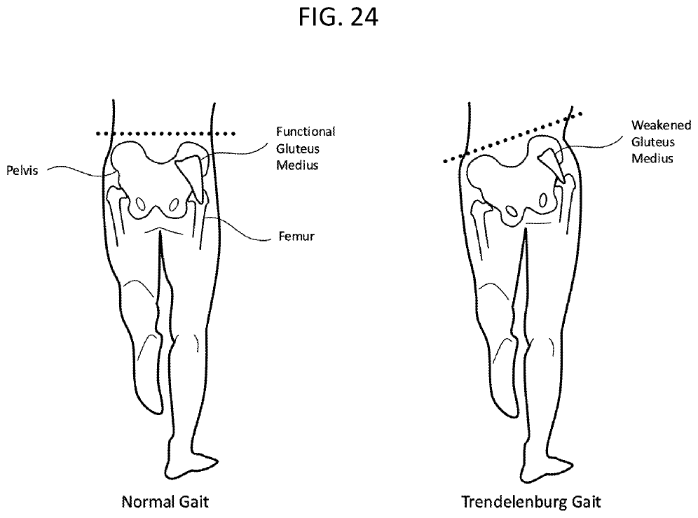

FIG. 24 shows how Trendelenburg gait manifests in a person's gait. The gluteus medius is connected to the pelvis and the greater trochanter of the femur. When the gluteus medius contracts, it causes the femur to rotate about the femoral head which tries to pull the leg into abduction. When the leg is in contact with the ground it cannot move (or movement is impeded) into abduction; instead, the opposite side of the pelvis is lifted.

The gluteus medius is, among other things, the muscle that keeps the pelvis level when weight is removed from one of the legs. If the gluteus medius is unable to support the weight of the pelvis (or is otherwise compromised) when the opposite leg is lifted off the ground, the pelvis will rotate down. To maintain their center of gravity over the leg in contact with the ground, the person will arch their back towards that leg. This exaggerated movement of the torso (and drop of the opposite hip) defines Trendelenburg gait.

The novel hip orthosis described herein augments the gluteus medius muscle and aids in lifting/supporting the pelvis. In aspects, it works by providing a tensile force (in cases, similar to the contraction of a healthy gluteus medius muscle) between the torso and the upper leg of the wearer.

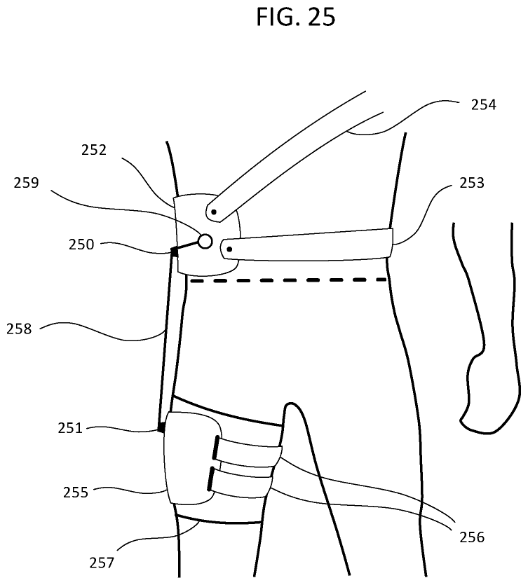

FIG. 25 shows a front view of one aspect of the present invention. The dashed lines show the approximate location of the pelvic shelf: the top of the pelvis bone. The waist component (252) shown in FIG. 25 can be secured to the wearer so that it preferably does not migrate downwards when the tensile force is applied. As illustrated, the waist component rests on the pelvic shelf above the hip joint and a waist strap (253) holds the waist component in place. An optional shoulder strap (254) can cross the torso and rest on the opposite shoulder and may be used to help prevent the downward migration of the waist component.

As illustrated, a thigh component (255) can be secured to the wearer's upper thigh below the hip joint with straps (256). To prevent upward migration and/or the rotation of the bottom edge away from the leg, the inner surface of the thigh component can be fitted with a high friction material—for example, silicone. Alternatively or in combination, a stretchable wrap (257)—for example, neoprene-may be wound around the leg before donning the thigh component and then the thigh component can be secured to the wrap-with, for example, Velcro/hook-and-loop fastener(s).

A connection component (258) bridges the waist component and thigh component and can be used to transmit the tensile forces between the waist and thigh components. The connection component can be attached to the waist component at a first anchor mount (250) and can be attached to the thigh component at a second anchor mount (251). As shown in FIG. 25, the connection component comprises a cabling system and a tightening element (259). The connection component can be hingedly attached to the waist component at a first anchor point and hingedly attached to the thigh component at a second anchor point. In general, the connection component preferably should at least span the distance between the first and second anchor points but may extend beyond.

The cabling system of the connection component can extend past the first anchor point on the waist component and terminate at the tightening element (259), which, in the embodiment shown in FIG. 25, can be comprised of a ratcheting dial and spool mechanism. In aspects, turning the dial and spool mechanism winds the cabling system about the spool thereby increasing the tensile force applied by the connection component.

Other non-limiting examples of tightening elements suitable for the connection component are a winding stick, a cam, a rack and pinion, a windlass, a spring, a piston, an elastic element, a pull tab, a screw, a lever, a block and tackle, a cam, memory wire, a motor, a linear actuator, and the like.

In FIG. 25, the ends of the connection component can be hingedly affixed to the first and second anchor mounts (250, 251). There can be one axis of rotation which can be perpendicular to the sagittal plane. In other embodiments, the anchor mount or anchor mounts may be affixed with a ball and socket joint to allow rotation in more than one axis. In yet other embodiments, the anchor mount can be a hole in the waist or thigh component. The cabling system of the connection component can be passed through the hole and knotted, crimped, tied, or the like, to prevent it from sliding out. This anchoring method (e.g., hole and knot) would also allow multiple axes of rotation between the connection component and the waist and thigh components. In another embodiment, the cabling system could be tied directly to the waist and/or thigh components at a post, loop, or similar feature.

The method of anchoring the connection component at the first anchor mount may differ than the method used at the second anchor mount.

In other aspects, the tightening element of the connection component can be attached to the thigh cuff. Alternatively, the tightening element may be positioned between the first and second anchor mounts. Alternatively, the connection component may comprise more than one tightening elements which are located at different positions.

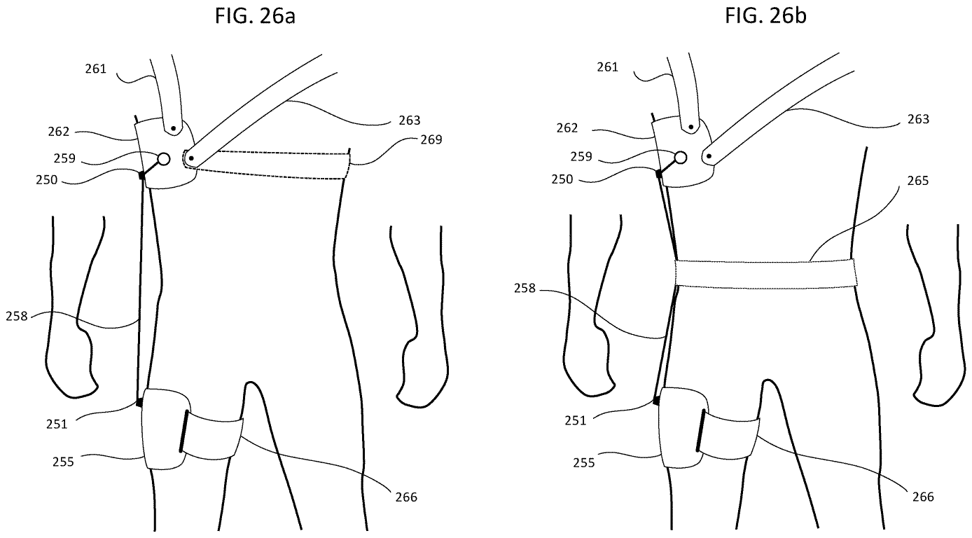

In another aspect, the waist component can be replaced with a chest component as shown in FIG. 26a. In aspects, the chest component (262) can conform to the side (or part of the side) of the torso above the waist and can be held in place with shoulder straps (261, 263) that can loop or be positioned over, across, around, or under, one or both shoulders. An optional chest strap (269) can be used to further secure the torso component to the wearer and prevent migration. In FIG. 26a and FIG. 26b, dual thigh straps (256) have been replaced with a single, wider strap (266). The chest component can be advantageous for wearers with a wound or tenderness near their waist that prevents them from wearing the waist cuff shown in FIG. 25, by way of example.

FIG. 26b shows an optional accessory to the hip orthosis shown in FIG. 26a. Preferably, the tensile forces generated by the hip orthosis can be the same or similar in magnitude and direction to the forces generated by the contracture of the gluteus medius. For individuals whose chest size is substantially larger than the width of their pelvis, the direction of the tensile force applied by the hip orthosis may less than optimal or preferable. In such cases, in cases, an additional waist strap (265) may be worn to change the direction of the force vector applied by the hip orthosis to more closely imitate the gluteus medius muscle.

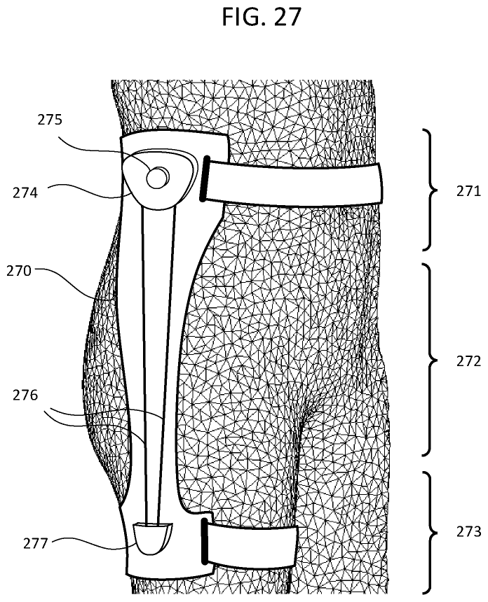

In aspects the hip orthosis may be a continuous, single piece. As shown in FIG. 27, the hip orthosis can be comprised of a continuous base (270) that spans the wearer's torso to the leg. The base can be made of flexible or semi-flexible materials such as cloth, synthetic fabrics, elastomers, rubbers, leather, neoprene, Lycra“, thermoplastic polyurethane (TPU), combinations thereof, and/or the like. The physical properties of the base may be similar throughout its length or may vary from position to position or location to location. For example, the base may be made of Lycra” with a piece of neoprene attached to the area corresponding to the waist of the wearer and another piece of neoprene attached to the area corresponding to the thigh of the wearer. The reinforced areas of the base, in this example, could withstand the tensile forces generated by the orthosis without bunching, whereas the unreinforced areas would remain relatively more flexible and conformable.

Although the base (270) can span the torso to the leg and be continuous, there can be a portion that corresponds to the torso component (271), a portion that corresponds to the thigh component (273), and a portion that corresponds to connection component (272), as described in, for example, FIG. 25, FIG. 26a, and FIG. 26b. For example, anchor mounts (274, 277) can attach to the waist portion and thigh portions, respectively. A tightening element (275) can adjust or be used to adjust the tensile force applied by the cabling system (276) between the thigh portion and the waist portion.

In aspects, placement of the tightening element on the waist portion allows the wearer to adjust the tensile forces applied while wearing the hip orthosis, although the tightening element does not have to be located on the waist portion, as other locations on the orthosis are contemplated.

In aspects, the cabling system (276) shown in FIG. 27 has two ends. The first end can be attached to the tightening element within the first anchor mount (274). The cable can extend to the second anchor mount (277) and loop through it, for example. The second end of the cabling system can also be attached to the tightening element. When the tightening element (275) is wound (or otherwise tightened or adjusted) to generate and/or increase a tensile force, such as an applied tensile force, the cabling system can, in embodiments, wind up on a spool (not shown) which pulls the thigh component portion (273) towards the waist component portion (272).

In another aspect, the second end of the cabling system can be attached to the first anchor mount. In cases where the tightening element is associated with or comprises a spool, by way of example, winding the spool by 1 unit can shorten the distance between the first and second anchor points by ½ units, thereby generating a 2:1 mechanical advantage in addition to any other mechanical advantage(s) caused by the tightening element's design, configuration, or location. The cabling system may be looped multiple times between the first and second anchor points to produce greater mechanical advantages (e.g., similar to a block and tackle, by way of example). Varying embodiments using examples of the mechanical advantage as explained herein can be used to increase or decrease a mechanical advantage imparted by the tightening element.

The hip orthosis shown in FIG. 27 can be advantageous, in cases, because it can be less confusing to don. For example, the wearer could rest the orthosis against the side of their torso and leg and secure the various straps.

In preferred embodiments, the tensile force(s) applied by the hip orthosis could be reduced, eliminated, blocked, or adjusted, when the wearer's afflicted leg is in the swing phase of the walking gait. When the afflicted leg is in the swing phase, the opposite leg is in the stance phase. Because the opposite leg is supporting or substantially supporting its own weight and the weight of the pelvis, the gluteus medius is not being used (or is being used less). The assistance to the gluteus medius provided by the hip orthosis may not be needed and, in fact, excessive tensile forces can disadvantageously alter the wearer's gait-abducting the swing phase leg needlessly.

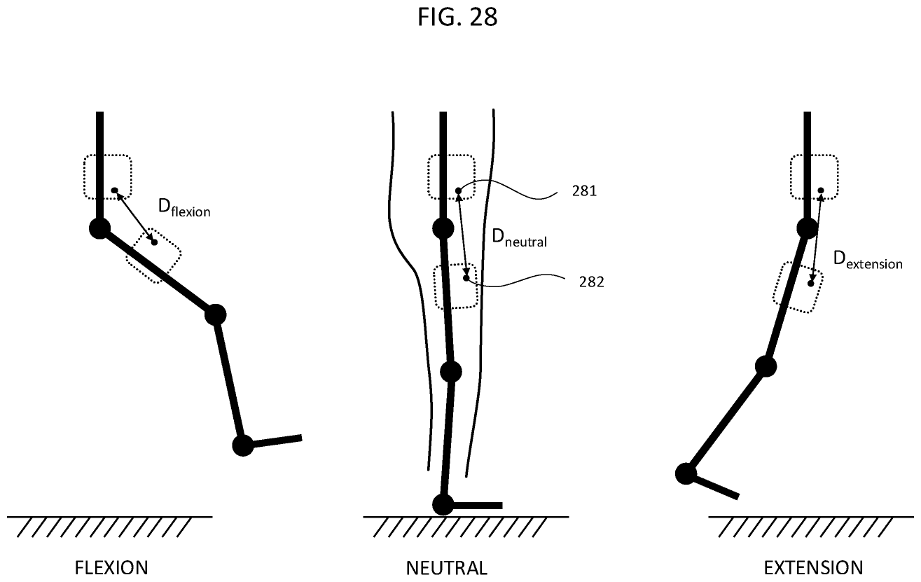

FIG. 28 is an illustration of the skeletal position of the human torso and leg in three different positions during the walking gait; heel strike/extension (labeled “flexion”), mid-stance/neutral (labeled “neutral”), and terminal stance/extension (labeled “extension”). Schematic illustrations of the waist component and thigh components of the hip orthosis are shown by the dashed lines in each of the three positions depicted in FIG. 28. The first anchor point (281) and second anchor point (282) are shown in FIG. 28 as located on the waist and thigh components. FIG. 28 shows how the direct line distance, D, between the first anchor point and the second anchor point changes with the position of the wearer's afflicted leg during the gait cycle. As depicted in the illustration, Dflexion≠Dneutral≠Dextension. The change in distance and the part of the gait cycle where the change occurs depends on the positioning of the at least two anchor points. Other factors can affect the change in distance between the anchor points, such as, for example, a tendency of the wearer to rotate their afflicted leg by varying amounts during the gait cycle. As noted herein, wounds, sore spots, or tenderness (from, for example, bursitis) may prevent relatively optimal/preferred locations/positioning of the waist and/or thigh components (and therefore the first and/or second anchor point(s)).

It is therefore preferable, in some cases, to be able to change the amount of tensile force applied by the hip orthosis during the gait cycle. One method of accomplishing this, by way of example, is by allowing an anchor point(s) to move during the gait cycle to counteract the change in distance between the anchor points.

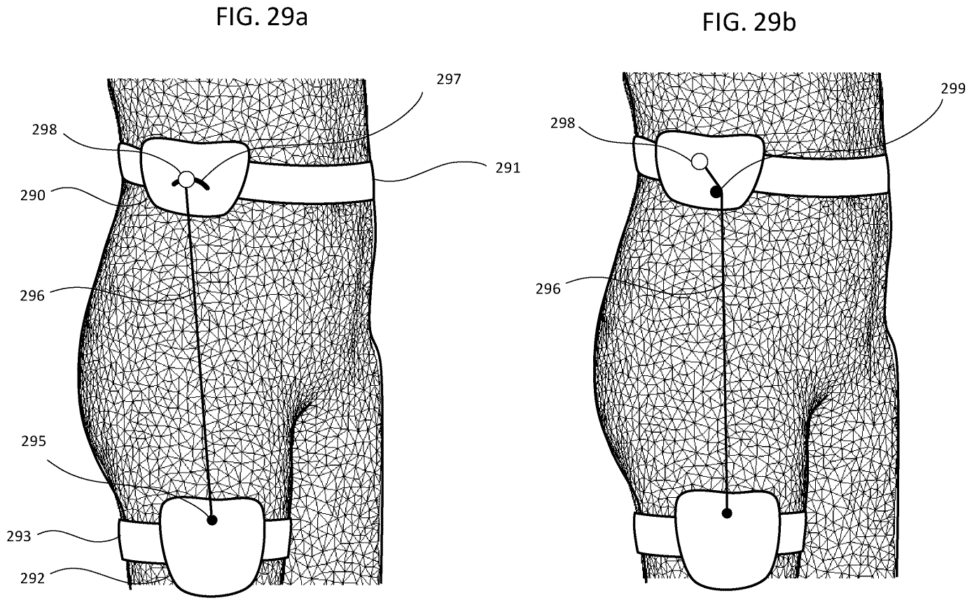

FIG. 29a shows another embodiment of the hip orthosis. In this embodiment, a waist component (290) can be secured to the wearer with a strap (291). A thigh component (292) can be secured to the wearer's leg with a strap (293). A connection component, in aspects, comprising a cabling system (296) and a tightening element (298), can bridge and/or otherwise operatively engage/connect the waist and thigh component. In aspects, the first end of the connection component can be anchored to the waist component by the tightening element (298). (In aspects of this embodiment, the tightening element (298) can serve a dual purpose; allowing for adjustment of the tensile force that pulls the waist component towards the thigh component, and acting as an anchor mount between the connection component and the waist component.) In aspects, the connection component can also be attached to the thigh component at an anchor mount (295). In this example, the second end (or thigh end) of the connection component can be attached to the thigh component at a thigh anchor mount (295), by way of example. In aspects, the tightening element (298) can ride or otherwise be located or positioned in a track (297) on the waist component. As the afflicted leg moves from heel strike to terminal stance (e.g., flexion to extension, respectively), the tightening element can slide or otherwise move from an anterior end of the track to a posterior end of the track. In aspects, the track is curved, and the curved nature of the track can be designed to force the distance between the anchor points to be the largest or relatively large (and therefore generate the most or relatively larger tensile force(s)) when the leg is in the neutral position, which occurs at mid-stance in the walking gait.

FIG. 29b shows an alternative embodiment to FIG. 29a, wherein the tightening element/anchor mount (298) is fixed to the waist component. A post (299) or protrusion can be placed on the waist component located on a posterior side of the cabling system of the connection component. When the afflicted leg is in extension or neutral (positions, such as of a walking gait), the cabling system can catch on or otherwise operatively engage with the post/protrusion to create an extra distance that the cabling system needs to travel between the waist anchor mount (298) and the thigh anchor mount (295). When the afflicted leg is in flexion (e.g., during the swing phase), the cabling system can move in front of the post/protrusion, causing a shortening in the distance the cabling system needs to travel between the first and second anchor points. In this manner, in aspects, the cabling system distance (and therefore tensile force(s)) is greatest or relatively greater during portions of the stance phase, and reduced in the swing phase.

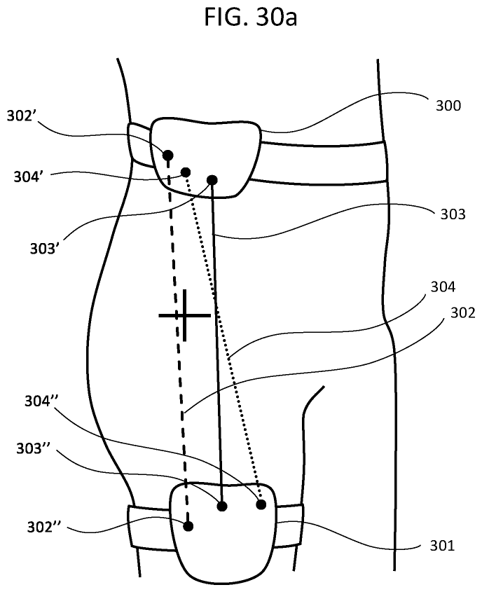

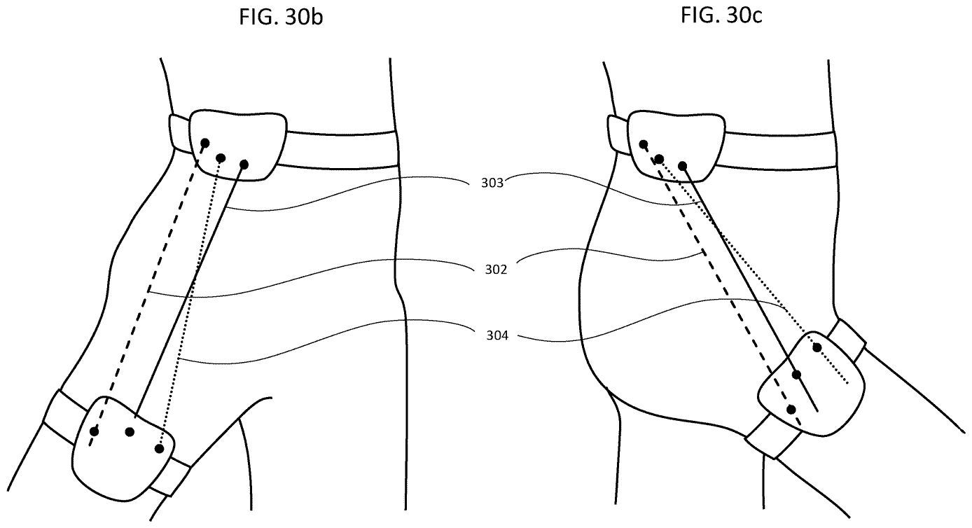

FIG. 30a shows an alternative embodiment that uses multiple inextensible cabling systems and multiple anchor points. A waist component (300) can have three anchor points (302′, 303′, and 304′). A thigh component (301) can have three anchor points (302″, 303″, and 304″). Three connection components (302, 303, and 304) can bridge the waist component and the thigh component. (In FIG. 30a, the connection components are shown comprising only a cabling system; no tightening system is shown, although it is envisioned as explained herein.) Connection component (303) is shown as a solid line. Connection component (302) is shown as a dashed line. Connection component (304) is shown as a dotted line. The center of rotation of the hip is shown by the crosshairs. In aspects, the tightening system(s) can be adjusted such that tension in each of the three connection components are the same.

FIG. 30 b shows the location of the thigh component when the hip is in approximately 25 degrees of extension. In this example, the solid, dashed, and dotted lines representing the three connection components have been rotated the same amount and lined up with their respective anchor points. As shown in FIG. 30b, the length of the cabling system of connection component (302) is longer than the distance between anchor points (302′ and 302″) (see also, FIG. 30a). The length of the cabling system of connection component (303) is shorter than the distance between anchor points (303′ and 303″) (see also, FIG. 30a). The length of the cabling system of connection component (304) is the same as the distance between the anchor points (304′ and 304″) (see also, FIG. 30a).

In embodiments, when the wearer moves from the position shown in FIG. 30a to FIG. 30b, the tensions transition from equally applied in FIG. 30a to predominantly applied by connection component (303). There can be both a change in magnitude (e.g., an increase) and direction of the summed force vectors from connection components (302), (303), and (304).

FIG. 30 c shows the location of the thigh component when the hip is in approximately 30 degrees of flexion. The solid, dashed, and dotted lines representing the three connection components have been rotated the same amount and lined up with their respective anchor points, in this example. As shown in FIG. 30a, the lengths of all three cabling systems of the connection components are longer than the distance between their corresponding anchor points, again in this example. However, change in length is not the same, in this example. Connection components (303) and (304) have a greater amount of slack than connection component (302), which is now the predominate source of tensile force. There is both a change in magnitude (e.g., a decrease) and direction of the summed force vectors from connection components (302), (303), and (304).

With selected positioning of the anchor points, the tensile force generated versus the position in the gait cycle can be customized to an individual's unique gait and gluteus medius augmentation needs. In aspects the cabling system and anchor point pairs may be independent as shown in FIG. 30a-FIG. 30c. In other aspects, some or all of the cabling systems may be looped together or connected to each other. In an alternative aspect, some of the independent cabling systems may share common anchor points. In yet another aspect, some cabling systems may be connected and some anchor points may be shared.

If a preferable distribution of tensile force versus gait cycle phase cannot be achieved with a predominately non-extensible cabling system, it is possible to add an elastic element in-line with the cabling system to serve to dampen undesirable force peaks. An elastic element may also provide comfort to the wearer; as the gluteus medius muscle weakens throughout the day, the pelvis of the opposed leg will drop more and more and the side of the waist over the afflicted leg will lift-increasing the distance between the anchor points, stretching the elastic, and generating more tensile force. In this manner, the amount of tensile force provided by the hip orthosis can vary to match the changing needs of the wearer.

In another aspect, one or more sensors can be placed in or on the shoe of the foot of the afflicted leg. The one or more sensors can be used to determine when the foot of the afflicted leg is in the stance phase (for example by sensing the pressure between the shoe and the ground, sensing the proximity of the ground, sensing the lack of acceleration of the foot, and the like). When the leg is in the stance phase, the one or more sensors can directly or indirectly activate the tightening mechanism to generate the tensile force between the first and second anchor points. When the leg is in the swing phase, the sensor can directly or indirectly deactivate the tightening mechanism.

The activation/deactivation may be accomplished by means of a clutch that engages/releases the cabling system. Alternatively, the one or more sensors may activate a motor, servo, linear actuator, or the like, which generates the tensile force.

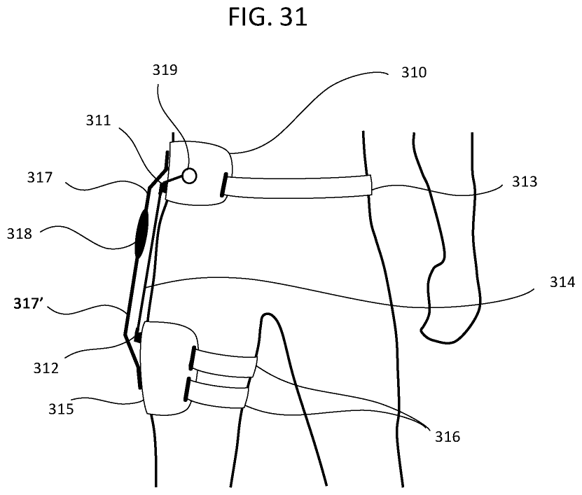

For some wearers, it is desirable to have ROM control for the hip joint in addition to the tensile forces described herein. FIG. 31 shows a variant of the hip orthosis shown in FIG. 25. A ROM hip brace mechanism, in FIG. 31 can be attached to the waist component (310) and the thigh component (315). A strap (313) can hold the waist component in place. As shown in FIG. 31, a pair of straps (316) can secure the thigh component to the leg of the wearer. A connection component (314) can join/connect the waist component and the thigh component and be attached at the anchor mounts (311) and (312). A tightening element (319) can connect to the cable system of the connection component. The ROM mechanism can be comprised of an upper strut (317) and a lower strut (317′) which are rotatably connected at, e.g., a disk (318). An additional pivot point or rotatably connection on the ROM hinge (not shown), having an axis perpendicular to the coronal plane, can allow the ROM hinge to abduct/adduct freely. Thus, the tensile forces applied by the connection component are still able to augment the gluteus medius muscle. The ROM mechanism disk (318) can have an axis of rotation that is perpendicular to the sagittal plane. Stops in the disk (not shown) can limit the range of extension and/or flexion.

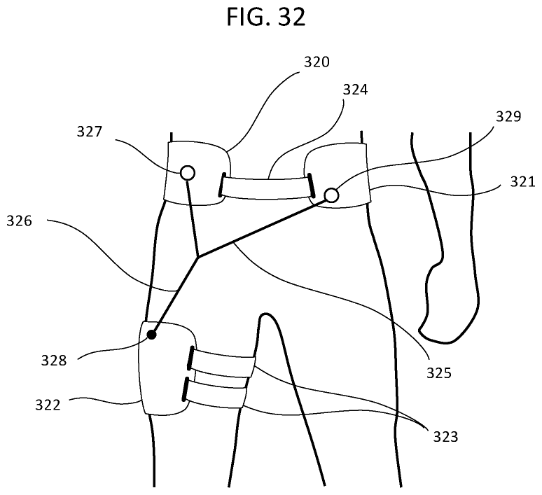

FIG. 32 shows an aspect that is configured to pull the wearer's leg into adduction (towards the midline of the body). A thigh component (322) is attached to the afflicted leg with, e.g., straps (323). A first waist component (320) is positioned above the hip joint of the afflicted leg. As described herein, the tensile forces generated by the orthosis can be relatively high, and to prevent migration of the waist component, it can be preferrable to wedge the first waist component against the pelvic shelf. A second waist component (321) is worn above the hip joint of the opposite leg. A waist strap (324) secures the first and second waist components to the wearer's torso. Optional shoulder straps (not shown) may be used to help offset migration forces if needed. A first connection component (326) bridges the first waist component and the thigh component. The first connection component is attached at a first tightening element (327), which can also serve as the anchor mount to the first waist component, and a second anchor mount (328) on the thigh component. A second connection component (325) bridges the second waist component and the first connection component. The first end of the second connection component is attached at second tightening element (329), which can also serve as the anchor mount to the second waist component. As shown in FIG. 32, the second end of the second connection component can be attached along the cabling system of the first connection component. Increasing the tension in the second connection component by means of the second tightening component (329) draws the cabling system of the first connection component towards the second waist component. This changes the force vector applied by the first connection component such that the afflicted leg is pulled into adduction.

As shown in FIG. 32, the cable systems and anchor mounts can be positioned on the anterior side of the first and second waist components and the thigh component. In other aspects, the cable systems and anchor mounts can be positioned on the posterior side. In yet another aspect, there are multiple cable systems and anchor mounts wherein some cable systems and anchor mounts are positioned on the anterior side and some are positioned on the posterior side. The benefit of multiple cable systems and anchor mounts can be that the direction of adduction can be controlled more finely, in aspects.

The various features described in the aspects corresponding to FIG. 25-FIG. 32 for a Trendelenburg hip orthosis can be applied to the adduction hip orthosis.

In aspects, the connection component can be configured to provide a compressive force by means of a distraction mechanism instead or in addition to a tensile force. A tensile force draws the waist component towards the thigh component. A compressive force separates the waist component from the thigh component.

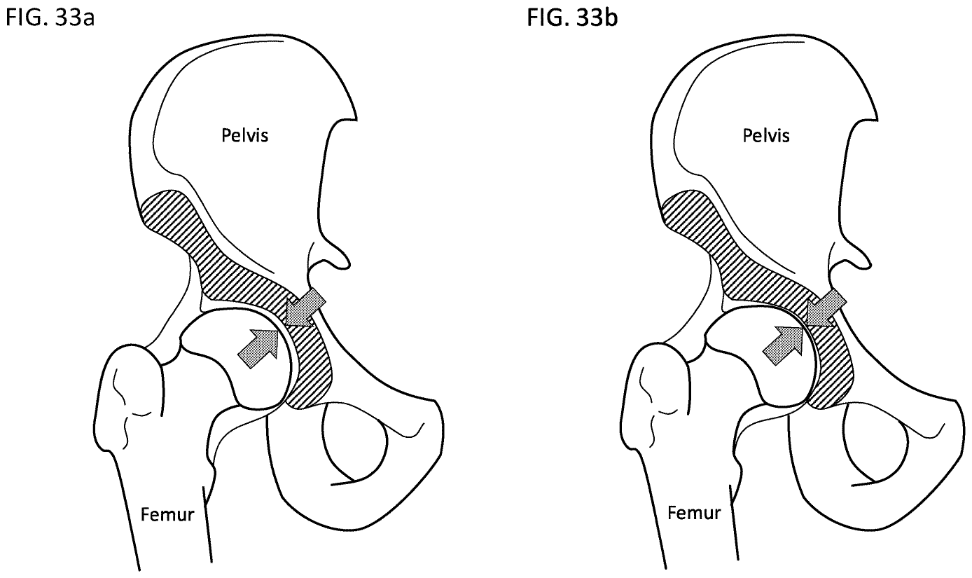

FIG. 33a and FIG. 33b show schematic views of the femoral head in the pelvic socket. FIG. 33a represents a normal hip joint. FIG. 33b represents a hip joint with degenerative OA. The distance between the arrows indicates the width of the cartilage that cushions the femoral head. As seen in FIG. 33b, the gap has narrowed allowing bone on bone contact.

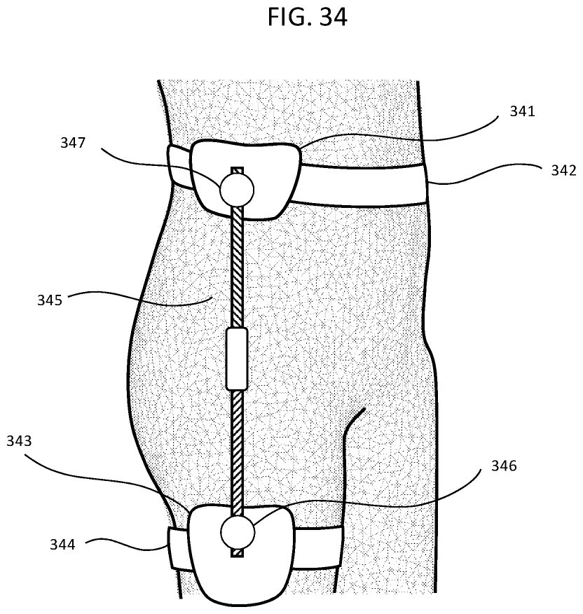

FIG. 34 is a front view of a distracting hip orthosis. A waist component (341) is secured around the torso of the wearer with a waist strap (342). A thigh component (343) is secured to the upper leg of the wearer via a thigh strap (344). A distraction mechanism (345) connects the thigh component and the waist component at anchor mounts (345, 347). When the distraction mechanism is activated, it generates a compressive force that pushes the thigh component and waist component apart. This compressive force unloads some of the weight of the upper body that pushes the pelvic socket and femoral head together.

If the compressive force is strong enough it will open the gap between the femoral head and the pelvic socket. In cases where the force isn't strong enough to open the gap, it will still reduce the force of the femoral head pushing into the pelvic socket thereby alleviating the pain associated with the diminished cushioning of hip joint cartilage due to OA.

Means of creating a joint distraction force have been disclosed in US 12,150,880 by Johnson et al. and incorporated herein by reference. In FIG. 34, the connection component comprises a distraction mechanism (345) which is represented by a turnbuckle. The turnbuckle has a pair of right-handed and left-handed threaded rods connected end-to-end. The threaded rods are held with correspondingly threaded nuts in the anchor mounts (345, 347). When the turnbuckle is rotated in one direction, the threads of the turnbuckle draw the nuts together. When the turnbuckle is rotated in the opposite direction, the nuts—and thereby the waist and thigh components—are pushed apart. This, in turn, generates an unloading force on the hip joint. A turnbuckle is an example of a distraction mechanism; other methods are disclosed in U.S. Pat. No. 12,150,880 such as eccentric cams, gears with variable radii, compound hinges with two or more pivot points, rack and pinion mechanisms, extendable sliding mechanisms, pistons, jack screws, linear actuators, levers, 4 bar linkages, and the like. The distraction mechanisms may be manually activated (e.g., by rotating the turnbuckle in FIG. 34) or powered by a motor, servo, pump, or the like and an appropriate energy source.

The anchor mounts (345, 347) are hingedly attached to the waist and thigh components (341, 343) with a rotation axis normal to the sagittal plane. This allows the leg wearing the thigh component to move into flexion and extension, but the pivoting motion of the two anchor mounts doesn't exactly match the rotation of the femoral head in the pelvic socket and is not ideal.

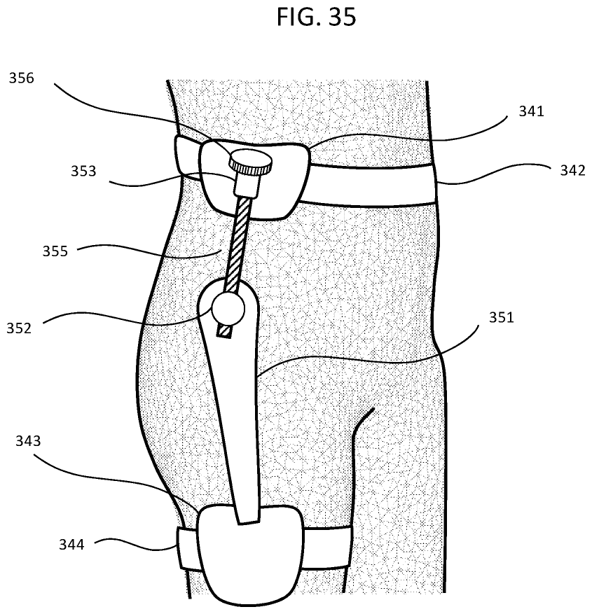

FIG. 35 is another aspect of the distracting hip orthosis. A waist component (341) is secured around the torso of the wearer with a waist strap (342). A thigh component (343) is secured to the wearer's leg with a thigh strap (344). A rigid extension (351) is attached to the thigh component. A distraction mechanism (355) connects the waist component and the extension of the thigh component at a fixed anchor mount (353) on the waist component and a pivoting anchor mount (352) on the thigh extension. The axis of rotation of the pivoting anchor mount is normal to the sagittal plane and, as shown, is in-line with the hip ball and socket joint which is ideal. In FIG. 35, the distraction mechanism (355) comprises a threaded rod, dial, and matching nut. The nut is affixed or integral with the pivoting anchor mount (352). The dial (356) is connected to the threaded rod which passes through the fixed mount (353). Turning the dial rotates the threaded rod drawing the pivoting anchor mount closer or pushing it farther from the waist component. In an alternate aspect, a mechanism that generates a mechanical advantage (e.g., a planetary gearbox) may be inserted between the dial and the threaded rod. This would enable a wearer with normal hand/wrist strength to generate a greater distraction force.

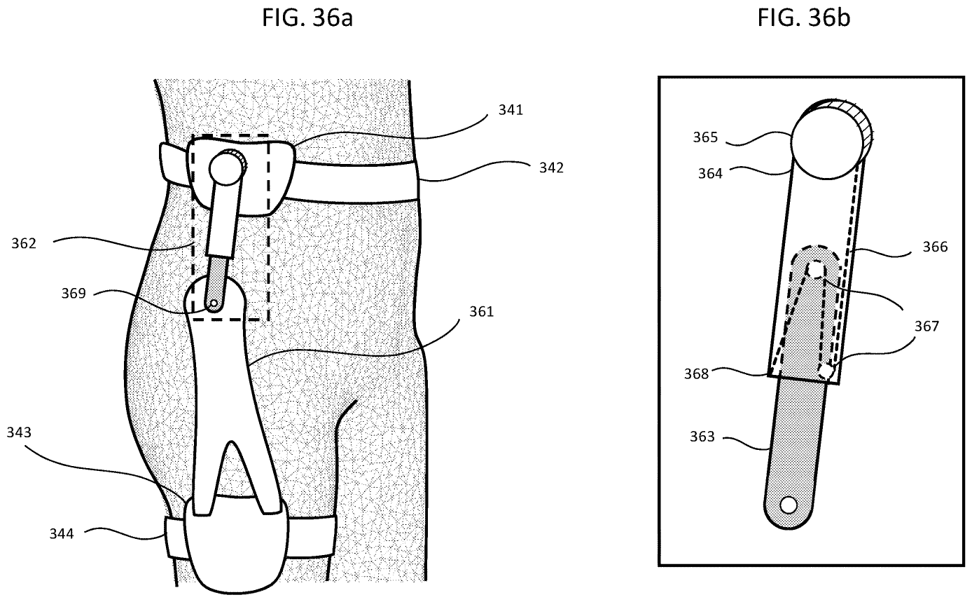

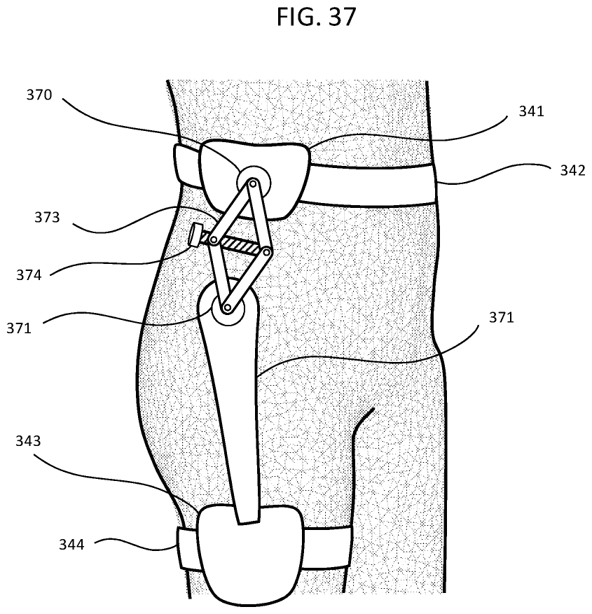

FIG. 36a shows a distracting hip orthosis that uses an extendable slide. A waist component (341) is secured around the torso of the wearer with a waist strap (342). A thigh component (343) is secured to the wearer's leg with a thigh strap (344). A rigid extension (361) is attached to the thigh component. A distraction mechanism (362) is attached to the waist component and hingedly attached to the rigid extension at a pivot (369) roughly along the axis of rotation of the hip joint in the transversal plane. FIG. 36b is a detailed view of the distraction mechanism (362).

A dial with a spool (365) is connected to a hollow slide (364). A shaft (363) slides inside the hollow slide and partially extends into it. A braided cord (366) is connected to the dial and spool on one end, wraps around two pulleys (367) and is secured at the slide at the opposite end (368). When the dial and spool is turned to collect the braided cord, the tightening of the cord forces the shaft to extend further outwards from the slide. This creates a distraction force that pushes the thigh component and the waist component apart reducing the bone-on-bone pressure of the hip joint.

FIG. 37 shows a distracting hip orthosis that uses a four-bar linkage. A waist component (341) is secured around the torso of the wearer with a waist strap (342). A thigh component (343) is secured to the wearer's leg with a thigh strap (344). A rigid extension (371) is attached to the thigh component. A four-bar linkage (373) is hingedly connected to the waist component at a first pivoting component (370) and hingedly connected to the rigid extension at a second pivoting component (371). A threaded rod with a knob on the end (374) is connected to the four-bar linkage. Turning the knob rotates the threaded rod in a nut (not shown) and scissors the four-bar linkage closed (or pushes it open, depending on the direction of rotation). In this manner, a distraction force can be generated between the waist component and the thigh component. An advantage of such a four-bar linkage is that it can generate a large force yet is not back-drivable.

Other means of generating a distraction force as detailed in US 12,150,880 can be employed between a waist component and a thigh component (or components attached to a waist component or thigh component). The distraction mechanisms described above and in U.S. Pat. No. 12,150,880 have the advantage that they can be adjusted by the patient while wearing the orthotic. This allows the patient to increase the distraction force while exercising or at the end of the day when their muscles tire or decrease it when it is not appropriate (e.g., while sitting down).

In aspects, it may be desirable to cover, protect, or hide the tightening elements and/or distraction mechanism and the sub-units (for example, the cabling system) of the connection component. The connection component may comprise a flexible sleeve made of cloth, neoprene, or the like. In aspects, the sleeve is attached to the waist and thigh components with Velcro™, glue, tied elements, sewn elements, or the like. In other aspects, the connection component may comprise a rigid or semi rigid cover or panel that articulates such that abduction/adduction of the afflicted leg is not inhibited.

In aspects, the connection component may comprise an elastic element to smooth out the application of tensile or compressive forces generated between the waist and thigh components in the various gait cycle stances. The elastic element may comprise by means of a non-limiting example: a spring, a piston, an article made of rubber, an article made of an elastomer, a leaf spring, and the like or combinations therein.

In aspects, the connection component may comprise a powered element to generate tensile or compressive forces generated between the waist and thigh components. The powered element may comprise by means of a non-limiting example: a motor and electrical source, a piston and compressed gas (or fluid) source, a piezoelectric material and an electrical source, a thermomechanical material and a thermal source, and the like or combinations therein. The powered element can be activated by, for example, a switch to turn it on or off, the powered element can be activated by, for example, a timer to adjust the tensile and/or compressive force generated based on the time of day/length of time worn; the powered element can be activated by a sensor, for example, an accelerometer to adjust the amount of tensile and/or compressive force applied based on the position of the leg in the walking cycle.

In aspects, the anchor mount or anchor mounts may be attached to a variety of different anchor points on the either waist component and/or thigh component for the purpose of changing the direction of tensile or compressive force generated by the connection component. In aspects, the anchor point may be a fixed position (e.g., selected by a clinician to customize the hip orthotic function to a particular patient), a moving position (e.g., a slide in which the anchor mount can move such that the anchor point position moves while the orthotic is worn), and/or a driven position (e.g., the anchor mount is located on linear actuator which drives the anchor point to a predetermined position based on, for example, the stage of the walking gait the wearer is current in.) In aspects, multiple anchor points may be provided on the waist component and/or the thigh component. The multiple anchor points may be located at a mixture of fixed, moving, or driven positions.

Sacroiliac joint pain is often a co-morbidity associated with hip joint disorders that may be caused by muscle imbalances, OA, trauma, and so on. The various aspects of the hip orthosis described herein may be advantageous in treating sacroiliac joint pain by supplementing muscle imbalances and/or distracting the pressure on the sacroiliac joint.

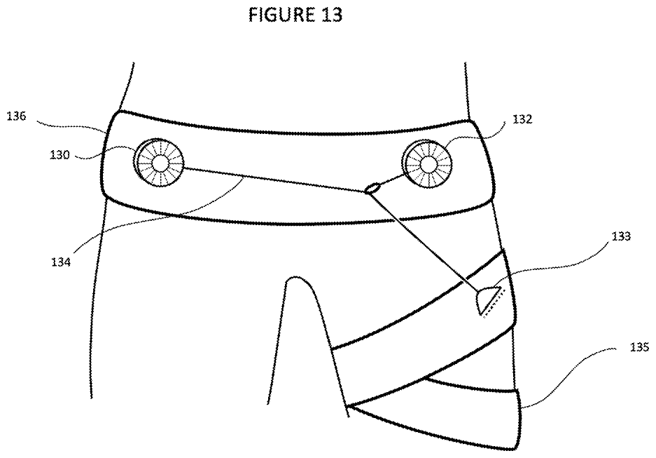

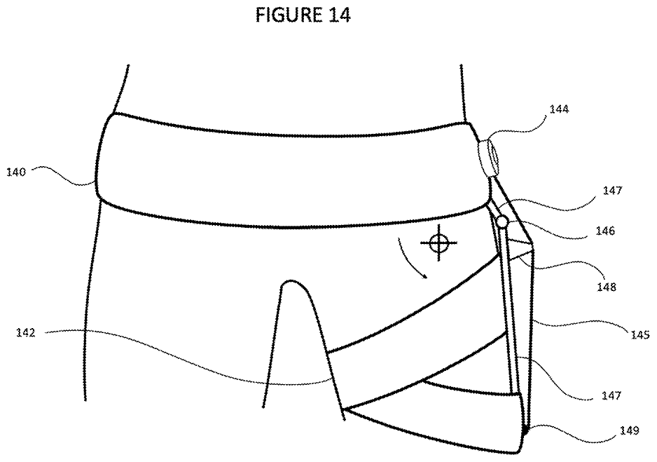

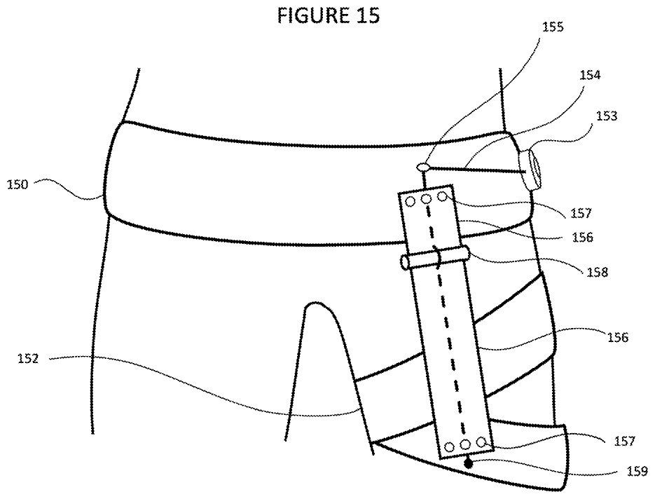

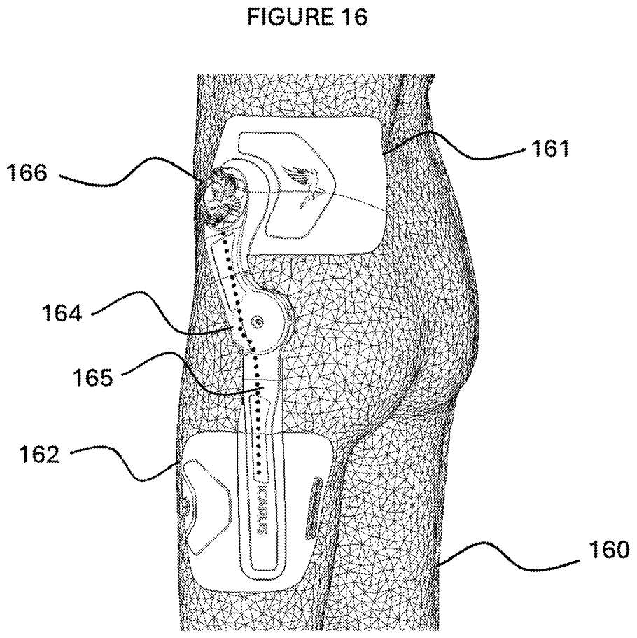

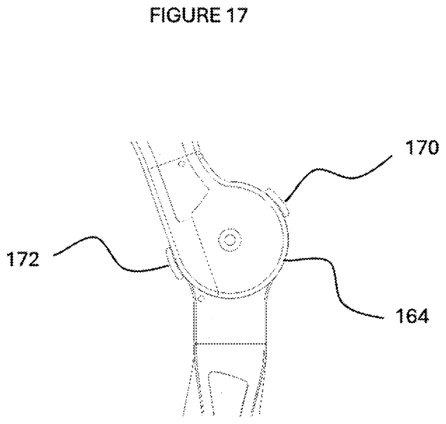

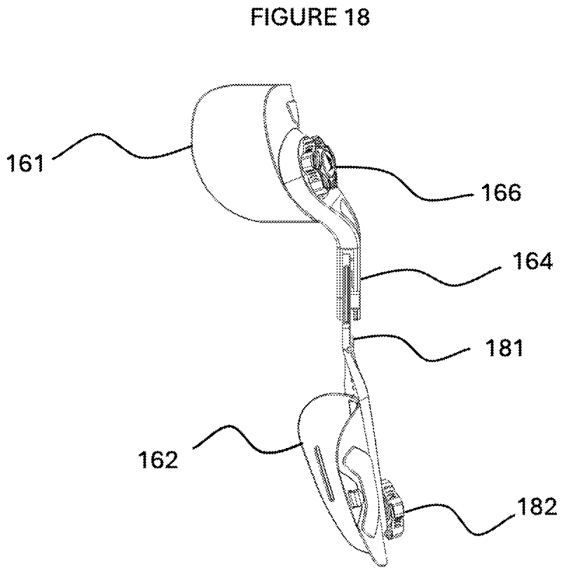

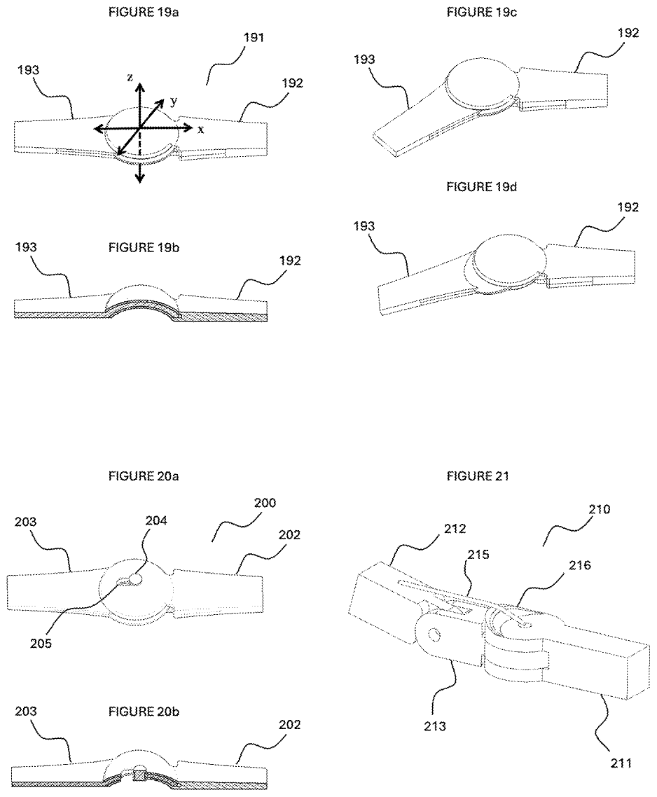

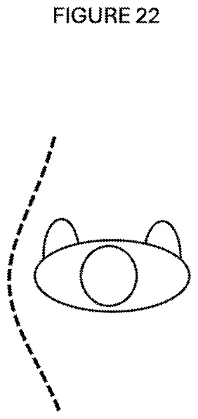

In aspects, the vector direction element (148) can be used to change the magnitude of the force applied by the tension in the cable in addition to, or in place of, altering the direction of the force vector. As shown in FIG. 14, moving the cable further away from the center of the femoral ball (as indicated by the crosshairs in FIG. 14) will increase the rotational moment arm generated by the tension in the cable.

As illustrated in FIG. 14, the vector direction element (148) is secured to the orthotic however, in some aspects element (148) may be loosely connected or entirely separate from the orthotic and held in place by frictional forces. In aspects, the vector direction element (148) may comprise a pad that is placed between the flexible cable (145) and the wearer's thigh. Such a pad would disperse the forces generated by the cable onto a larger area of the wearer's thigh improving comfort while the orthotic is being worn. The thickness of the pad itself would provide the increase to the rotational moment arm. The pad may further comprise elements that guide the cable, secure the cable, protect the pad from abrasion generated by the cable, dampen any sound caused by the cable rubbing on the pad, or combinations thereof and the like.

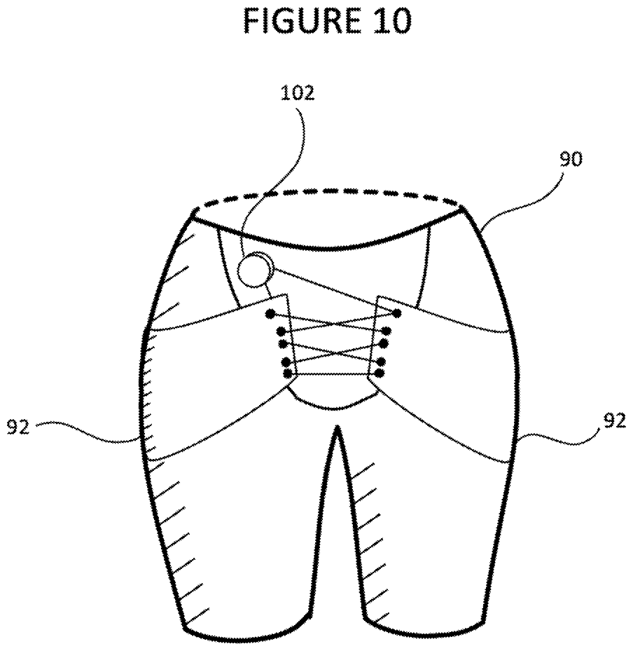

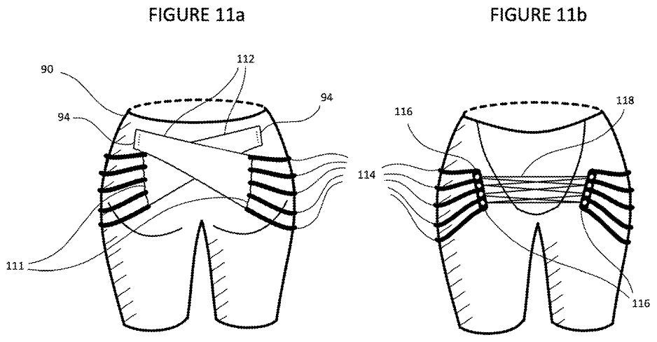

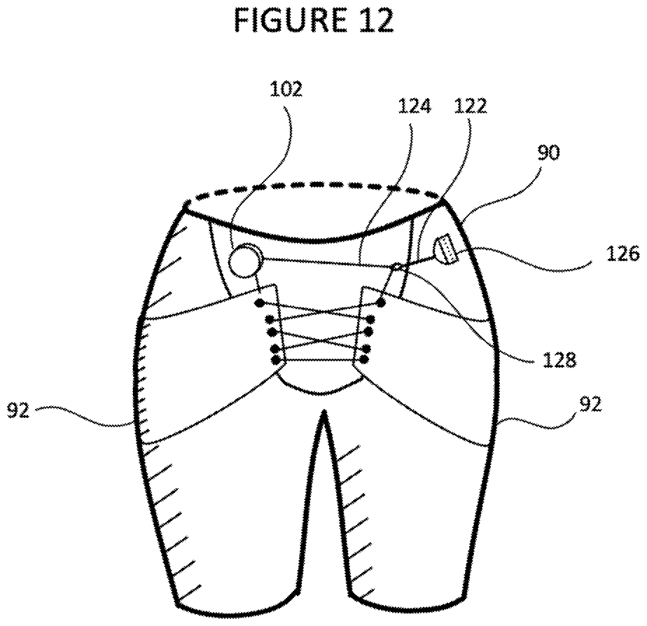

In aspects, elements of a form fitting brace as shown in FIG. 10 can be combined with elements of a joint distraction brace as shown in FIG. 14. In a non-limiting example, a distraction hip brace may comprise a thigh component, a waist component, a distraction mechanism, and an optional shoulder component. The distraction mechanism connects the thigh component and the waist component and generates a distraction and/or unloading force across the hip joint. The distraction mechanism may comprise a flexible cable, a tensioning element, and anchor point(s) as shown in FIG. 14 or may comprise other distraction mechanisms as described below. The distraction force needed to unload the hip joint may be quite high and pull the waist component towards the thigh component until wearing the orthosis is uncomfortable. An optional shoulder strap that connects to the waist component can offset some of the forces pulling the waist component downwards to the neck and shoulder of the wearer.

Donning an orthotic that has three components (a thigh component, a waist component, and a shoulder component) can be cumbersome and so in some aspects it is advantageous to combine some or all of the components into a single, form fitting garment similar to the form fitting brace shown in FIG. 10. In a non-limiting example, the waist component and the thigh component can be joined as a single, form fitting garment where the waist portion of the garment is donned first by stepping through or wrapping it around the waist. The thigh portion may then be secured by stretching and wrapping it around the thigh securing it in place with, for example, a Velcro™ strip. If a shoulder strap is part of the orthotic, the shoulder portion and waist portion could be donned like putting on a tee-shirt followed by securing the thigh portion as discussed above. The distraction mechanism may be sewn inside the garment with only the tensioning element visible. As needed, stiff, rigid, or semi-rigid components can be incorporated in the garment where extra support is required. (As a non-limiting example, a swatch of leather can be sewn into/onto the garment at the thigh portion to anchor one end of the cable (145) when using a distraction mechanism as shown in FIG. 14.) The resulting hybrid orthotic would have the form-fitting advantages of the embodiment shown in FIG. 10 with the unloading distraction features of the orthotic shown in FIG. 14.

Generating distraction forces across a joint is an effective way to reduce bone-on-bone contact caused by osteoarthritis. The embodiment shown in FIG. 14 is one method of generating a distraction force between the femoral ball and the hip socket.

In aspects it may be desirable to generate a constant or nearly constant distraction force. For example, while standing still the weight of the torso pushes the femoral ball into the hip socket which can cause pain. A distraction orthotic may be used to overcome this pain be generating a constant (or nearly constant) distraction force to off-load the weight of the torso.

In other aspects it may be desirable to generate a distraction force that varies with the activity or stance of the wearer. For example, patella femoral osteoarthritis (PFOA) in the knee is particularly painful when the knee is bent and bearing weight (such as when climbing stairs). A distraction orthotic for (PFOA) would benefit from applying a larger distraction force when the knee was bent than when the knee was straight.

Of course in some situations it is desirable to combine these features into a single orthotic. For example, a hip brace could have a constant distraction force to offset the weight of the torso and an additional distraction force applied when the opposite leg is in the swing phase of the walking gait (and all the weight of the torso is borne by the leg wearing the hip brace).

In yet other aspects it may be desirable to generate a long-term distraction force (for example, greater than several hours per day) for the purposes of healing damaged cartilage in the distracted joint. Said long-term distraction force may be milder than an activity-based distraction force to promote comfort while wearing the orthotic, for example, while sleeping. The long-term distraction orthotic may be configured differently than an unloading distraction orthotic. As a non-limiting example, an unloading hip brace may be configured to provide maximum distraction force when the hip is fully extended (standing or weight bearing) whereas a long-term distraction hip brace may be configured to provide maximum distraction force while the wearer is sitting.

There are benefits of joint distraction for regrowing cartilage. The benefits of knee joint distraction (KJD) surgery in regenerating knee cartilage has been demonstrated. KJD surgery involves a securing a cage-like structure to the tibia and femur and putting the joint in traction for 6-8 weeks to open the knee joint by ~5 mm. It was suggested that the increase in joint spacing had allowed for greater synovial fluid transport which may have aided in cartilage regeneration.

KJD surgery has risks. The cage-like structure pierces the leg muscles in order to screw into the bones and are potential sites of infection. In addition, damage to the bone where the structure is secured may make future total knee replacement surgery more difficult than otherwise. Some patients who have successfully undergone KJD surgery have stated that they would not do it again due to all the unpleasant aspects of the procedure.

An orthotic that can provide long-term joint distraction would be desirable even if the healing process took longer than the 6-8 weeks required by the surgical procedure. Risks of infection or adverse reactions to anesthesia would be eliminated. There would be no damage to the bones that make up the joint and thus no concerns for future surgeries. Adoption rates would likely be higher and the benefits to society would be greater.

In KJD surgery, the forces are applied directly to the bones in the joint. (In fact, the forces aren't measured; the amount of space generated-the distraction-is measured.) In contrast, a long-term joint distraction orthotic (or a joint healing distraction orthotic) applies a distraction force to the wearer's skin, adipose tissue, and muscle which is transferred to the bones in the joint. Laxity between the connective tissues and the bone requires the orthotic to generate a greater amount of distraction than the actual desired bone-to-bone distraction. For this reason, the distraction distance that a joint healing distraction orthotic is capable of generating may be greater than the distraction distance needed for an unloading distraction orthotic. Anti-migration requirements (devices or strapping methodologies that inhibit the orthotic from sliding along a limb) may be different than for unloading distraction orthotics for the same reason.

In FIG. 14., the dial (144) allows the wearer to select the amount of joint distraction that they desire. For example, the wearer may select one level of tension in the cable (145) while standing and a different level of tension while walking. The embodiment shown in FIG. 14 is a partial or directional unloading distracting orthotic that relieves pain, enhances range of motion, or both.

A joint distraction orthotic that is primarily designed for long-term wear to heal the joint is comprised of first component, a second component, and a distraction mechanism. The first component is worn on a first side of the joint, the second component is worn on the second side of the joint, and the distraction mechanism pushes, pulls, twists, etc. the first component away from the second component thereby creating a distraction force across the joint.

The first and second component may further comprise anti-migration elements to prevent slippage such as, but limited to, high friction materials, anatomically shaped shells that cup the limb, straps, elastics, compression wraps, elements that are adhesively secured to the wearer's skin, wedges, clamps, sleeves, self-tightening elements, combinations thereof and the like.

The distraction mechanism may further comprise a screw, a jack-screw, turnbuckle, a cam, a tensioning element, an extension element, a rack and pinion, a pulley, a block and tackle, a wedge, a linkage, a four-bar linkage, a linear actuator, worm gear, motor, spring, elastic element, pneumatic cylinder, hydraulic cylinder, combinations thereof and the like.

The distraction mechanism or mechanisms may be distributed about the circumference of the joint more or less symmetrically to facilitate uniform distraction of the joint. In other aspects the distraction mechanism or mechanisms may be distributed non-symmetrically around the joint to facilitate non-uniform distraction of the joint. In other aspects, the strength and/or the throw of the distraction mechanism or mechanisms may be altered such that a symmetrical distribution produces a non-uniform distraction, or a non-symmetrical distribution produces a uniform distraction.

In aspects, the distraction mechanism or mechanisms can be adjusted by the wearer to produce a desired amount of distraction force. In other aspects, the distraction mechanism or mechanisms can be adjusted by the wearer to product a desired amount of distraction distance.

Sensors or indicators may be used in conjunction with the distraction mechanism or mechanisms to quantify the distraction force or distraction distance applied. In a non-limiting example, a transducer could be used to measure the force applied between the first component and the second component. In another non-limiting example, a spring with a known spring constant could move a needle on scale to indicate the amount of distraction force applied. In another non-limiting example, a reference mark could be placed on the wearer or an article worn by the wearer and the distraction mechanism or mechanisms could be adjusted until the reference mark was visible in a window located in the orthotic.

In other aspects, a sensor or indicator can be connected to a microcontroller which alerts the wearer when the distraction force or distance is out of bounds, logs the data gathered by the sensor or indicator, transmits the data gathered by the sensor or indicator, causes a motor or actuator to energize (or de-energize) based on data gathered by the sensor or indicator, combinations thereof and the like.

In aspects, the joint distraction orthotic may further comprise a safety component that prevents the distraction mechanism from generating a distraction force or distance that is undesirable. In a non-limiting example, the distraction mechanism may push against an anchor element on the first and second components. The anchor elements may be secured to the first and second components with break-away features or high-friction sliding features. If the distraction force exceeded the strength of the break-away features, the anchor would separate from either the first or second component. Likewise, if the distraction force exceeded the frictional forces of the sliding features, the anchors would slide along the sliding features. In both examples, the distraction force would be limited by the strength of the safety features.

In aspects, the safety features could also be used as a treatment protocol. In a non-limiting example, several sets of sliding features with different levels of friction may be provided. In the first stage of treatment using the joint distraction orthotic, the wearer may use a first set of sliding features with a specific frictional profile. After donning the joint distraction orthotic, the wearer would increase the distraction force until the sliding features first begin to slide thereby generating a known distraction force profile. After several weeks, the protocol may instruct the wearer to replace the first set with a second set of sliding features with a different specific frictional profile, and so on.

FIG. 35 represents an embodiment of a distraction hip orthosis, incorporating a dial-adjustable tensioning system capable of increasing the length of a vertical strut of the hip orthosis. In this embodiment, the orthosis may include a waist portion, a thigh portion, and a distraction mechanism extending therebetween, wherein actuation of the dial causes the vertical strut to lengthen and thereby urges the waist portion and thigh portion away from one another to generate a distraction and/or unloading force across the hip joint. The dial-adjustable system may permit the wearer or clinician to selectively increase, decrease, or fine tune the amount of distraction for standing, walking, rehabilitation, or extended wear, and may be used in conjunction with anti-migration elements, rigid or semi-rigid load-distribution members, and other adjustment features described herein. FIG. 36 represents an embodiment of a distraction hip orthosis similar in certain respects to the embodiments of FIG. 34 and FIG. 35, wherein the orthosis includes a waist and/or torso portion, a thigh portion, and a distraction mechanism configured to produce a controllable force that distracts, unloads, or otherwise reduces contact force between the femoral head and the acetabulum. In some aspects, the embodiment of FIG. 36 may incorporate one or more adjustable force-transmission, compression, or expansion elements, optional garment or form-fitting portions, and optional anti-migration or safety features so that the magnitude, direction, duration, or comfort of the distraction force can be tailored to the wearer's anatomy, pathology, activity level, or stage of recovery.

In additional aspects, the distraction orthosis can be configured such that the forces transferred between the waist portion and the thigh portion resolve into a distraction component across the hip joint even where the orthosis does not produce a large visible separation of the femoral head from the acetabulum. Stated differently, the orthosis may reduce contact force between articular surfaces by applying a force vector having a superior, lateral, anterior, posterior, or oblique component that opposes at least part of the compressive joint reaction force generated during standing, walking, stair climbing, sit-to-stand, or other weight-bearing activity. Thus, the term distraction, as used herein, includes complete distraction, partial distraction, directional unloading, selective unloading of a diseased region of cartilage, redistribution of contact pressure, reduction of peak compressive force, combinations thereof, and the like. In some wearers, the primary benefit may be a measurable increase in joint separation, whereas in other wearers the primary benefit may be reduced pain, improved range of motion, improved tolerance for ambulation, or reduced contact stress without substantial radiographically observable separation. In some embodiments, the direction and magnitude of the distraction force are determined at least in part by the relative positions of one or more anchor regions on the waist portion, torso portion, thigh portion, or combinations thereof. By way of non-limiting example, a more superiorly located waist anchor and a more inferiorly located thigh anchor may increase a generally axial distraction component, while anterior or posterior repositioning of one or both anchor regions may add a rotational component that biases the femur away from a particular region of the acetabulum. In some embodiments, multiple anchor regions are provided so that a clinician or wearer may choose among alternative pathways for a force-transmission member to customize the direction of unloading for a particular anatomy, pathology, stage of recovery, or activity. The anchor regions may comprise reinforced textile regions, sewn pathways, loops, guides, channels, rings, buckles, slots, hook-and-loop attachment regions, embedded plates, rigid or semi-rigid inserts, low-friction eyelets, combinations thereof, and the like. In some aspects, a force-transmission member can be re-routed between different anchor regions to change the force vector without replacing the orthosis. In further aspects, the orthosis may comprise a flexible garment portion that substantially continuously connects the thigh portion with the waist and/or torso portion. Such a flexible garment portion may comprise shorts, a sleeve, a compression garment, a pelvic garment, a textile panel, an elastic fabric, an inelastic fabric, or combinations thereof. The garment may include localized reinforced regions that distribute force over a larger area of the wearer and thereby improve comfort while resisting bunching, rolling, or migration. One or more rigid or semi-rigid load-distribution members may be coupled to, captured within, laminated to, inserted into, or otherwise embedded within the garment portion. Non-limiting examples include polymer stays, spring elements, composite battens, molded shells, leather or synthetic reinforcement panels, webbing capture zones, and rigid inserts positioned at anchor regions or at areas of anticipated high load. In this manner, a hybrid orthosis may retain the conformability and donning convenience of a garment while also providing structural pathways for transmitting clinically relevant distraction forces.

The distraction mechanism need not be limited to a cable tightened in tension. In some embodiments, the orthosis comprises one or more compression or expanding apparatuses that push orthotic portions away from one another, either directly or through intermediate linkage members, thereby producing distraction across the hip joint. Non-limiting examples include telescoping struts, screw expanders, jacks, turnbuckles, wedge mechanisms, cam mechanisms, rack-and-pinion mechanisms, pneumatic members, hydraulic members, resilient columns, spring-biased expansion members, and combinations thereof. A single mechanism may produce both tension and compression in different portions of the orthosis. Adjustment of such mechanisms may be accomplished by a buckle, cam buckle, ladder lock, ratchet, dial, reel, lever, pull tab, hook-and-loop fastener, winding member, motorized actuator, or combinations thereof. The adjustment mechanism may permit continuous adjustment, stepped adjustment, indexed adjustment, clinician-set adjustment, wearer-set adjustment, or activity-specific adjustment. In some embodiments, a wearer may increase unloading while standing or walking and reduce unloading while seated or during lower-demand activities. In some treatment protocols, the orthosis is configured for relatively short-term activity-based unloading to reduce pain during functional tasks, whereas in other protocols the orthosis is configured for extended wear to promote a more prolonged period of reduced joint compression. Prolonged or repeated unloading may facilitate a more favorable mechanical environment within the joint by reducing repetitive contact stress, permitting improved synovial fluid exchange, reducing irritation of sensitized structures, and allowing the wearer to maintain mobility with less pain. In still further aspects, the orthosis can be configured to provide a lower distraction force for extended wear, such as several hours per day or during rest, and a higher distraction force during weight-bearing activity. Safety features may include breakaway connections, torque-limiting clutches, slip interfaces, overload indicators, displacement stops, clinician-set maximum adjustments, sensors, microcontroller-based limits, or combinations thereof, so that the orthosis does not exceed a desired distraction force or displacement. These embodiments may be useful for conservative management of hip osteoarthritis, for post-operative recovery after hip surgery, or for other conditions in which reducing contact force at the femoroacetabular interface is desirable. Additional Abduction Assist Embodiments and Supporting Technical Rationale During single-leg stance, the hip abductor musculature, including the gluteus medius and gluteus minimus, acts to stabilize the pelvis and resist contralateral pelvic drop. Where the gluteus medius is weakened, painful, inhibited, surgically disrupted, or fatigued, the wearer may demonstrate pelvic drop, compensatory trunk lean, reduced

stance time, reduced walking speed, or other gait deviations commonly associated with Trendelenburg gait or Trendelenburg-like gait. An abduction-assist orthosis may supplement the moment ordinarily generated by the wearer's abductor musculature by transferring force between a torso component and a thigh component such that the resulting moment tends to abduct the femur and/or elevate the contralateral side of the pelvis during stance. In this manner, the orthosis may reduce mechanical demand on compromised tissues while preserving ambulation and may also serve as a rehabilitation aid while muscle performance is being restored. The need for abduction assistance can vary throughout the gait cycle and from wearer to wearer. In many cases, the greatest need for assistance occurs during portions of stance in which the affected limb bears most or all of the body weight, because those are the portions of gait in which pelvic stabilization demand on the hip abductors is greatest. Accordingly, in some embodiments the orthosis is configured to provide a greater abduction-assist moment during stance than during swing by reason of anchor geometry, variable cable path length, engagement features, elastic elements, sensors, clutches, powered actuation, or combinations thereof. In other embodiments, a lower level of abduction assistance may be maintained during both stance and swing to guide limb position, reduce fatigue, or improve neuromuscular training. By tuning the timing and magnitude of assistance, the orthosis may reduce contralateral pelvic drop, reduce compensatory trunk lean, improve gait symmetry, improve walking endurance, or combinations thereof. In additional aspects, the same orthotic platform may be configured to provide abduction assistance, distraction assistance, adduction assistance, range-of-motion control, or combinations thereof.

For example, a first force-transmission member may be routed to generate an abduction moment while a second force-transmission member or expanding member produces a distraction or unloading force across the hip joint. A ROM hinge or stop assembly may be included to limit flexion, extension, abduction, adduction, internal rotation, external rotation, or combinations thereof without substantially interfering with the desired assistive force. The magnitude of the abduction-assist moment may be altered by changing tension in a force-transmission member, changing the location of an anchor region, changing the position of a vector direction element, changing the distance of the force path from the center of hip rotation, or combinations thereof. In this way, the orthosis can be tuned to a wearer having abductor insufficiency alone, painful osteoarthritis alone, post-operative weakness alone, or overlapping conditions that benefit from both unloading and muscle-assist functions. Non-limiting examples of wearers who may benefit from abduction-assist embodiments include those with gluteus medius or gluteus minimus weakness, tendon injury or repair, superior gluteal nerve compromise, post-arthroplasty abductor insufficiency, post-operative weakness following hip preservation or reconstructive procedures, pain-inhibited abductor activation associated with osteoarthritis, and fatigue-related gait deterioration. The supporting rationale for such embodiments is that pelvic stability during single-leg stance depends on a sufficiently large abductor moment about the hip. Where a wearer cannot reliably generate that moment, an external orthotic moment can substitute for or augment the deficient muscular contribution. This external assistance may reduce compensatory movement patterns, permit more normal gait mechanics, and support rehabilitation by allowing graded assistance that can be increased, decreased, or phased out over time as symptoms or strength change.

Embodiments include a computer readable medium comprising one or more computer files comprising a set of computer-executable instructions for performing one or more of the calculations, steps, processes and operations described and/or depicted herein. In exemplary embodiments, the files may be stored contiguously or non-contiguously on the computer-readable medium. In aspects, the files or data may be sent directly or indirectly to the cloud or remote servers(s). Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers or involve a network of remote servers hosted on the internet. In aspects, local, edge, or remote computing possibilities are used to store, manage, and process data. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general-purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes, and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1) A hip orthosis comprising:
a torso component configured to be secured about a torso of a wearer of the hip orthosis;
a thigh component configured to be secured about a thigh of the wearer of the hip orthosis; and
at least one connection component comprising or connected to a first anchor mount attached to the torso component and comprising or connected to a second anchor mount attached to the thigh component;
wherein the at least one connection component is configured to transfer a tensile force between the torso component and the thigh component to augment a contraction force of a gluteus medius muscle of the wearer of the hip orthosis; and
wherein a magnitude and/or a direction of the tensile force is configured to be adjustable by the wearer of the hip orthosis while the hip orthosis is being worn.

2) The hip orthosis of claim 1 wherein the tensile force, which is applied or generated by the at least one connection component, is configured to pull a leg of the wearer of the hip orthosis into abduction and adduces the leg of the wearer towards of a midline of a body of the wearer of the hip orthosis.

3) The hip orthosis of claim 2, wherein the tensile force is applied or generated during a stance phase and a swing phase of a gait cycle of the wearer of the hip orthosis.

4) The hip orthosis of claim 1, wherein the at least one connection component further comprises a flexible element and a cabling system, the cabling system comprising a cable or cables.

5) The hip orthosis of claim 4, further comprising at least one adjustment component, wherein the at least one adjustment component is operative to adjust the magnitude and/or direction of the tensile force transferred by the at least one connection component, and wherein the at least one adjustment component comprises a dial, a spool, or both, upon which at least a portion of the cable or cables of the cabling system are wound around.

6) The hip orthosis of claim 4, further comprising a first and a second adjustment components, wherein the first adjustment component is operative to adjust the magnitude of the tensile force transferred by the at least one connection component, and the second adjustment component is operative to adjust a direction of the tensile force transferred by the at least one connection component.

7) The hip orthosis of claim 4, wherein the cabling system does not comprise a closed loop system.

8) The hip orthosis of claim 1, further comprising a waist component, wherein the waist component comprises a shoulder strap configured to prevent the waist component from migrating downwards when a tensile force is transferred between the waist component and the thigh component by the at least one connection component.

9) The hip orthosis of claim 1, further comprising a waist component, wherein the first anchor mount or a third anchor mount of the at least one connection component is affixed to the waist component posterior to the second anchor mount of the at least one connection component.

10) The hip orthosis of claim 1, further comprising a waist component and two connection components, a first connection component and a second connection component, wherein the first connection component is operative to transfer tensile force between the waist component and the thigh component, and the second connection component is operative to limit a range of motion between the waist component and the thigh component.

11) The hip orthosis of claim 1, further comprising a waist component, wherein the waist component and the thigh component are worn on a same side of a sagittal plane of the wearer of the hip orthosis.

12) The hip orthosis of claim 1, further comprising a flexible element, wherein the at least one connection component comprises an energy storage element in-line with the flexible element.

13) A hip orthosis comprising:
a first waist component operative to secure partially or completely around a waist of a wearer of the hip orthosis;
a second waist component operative to secure partially or completely around the waist of the wearer of the hip orthosis across a sagittal plane relative to the first waist component;
a thigh component operative to secure partially or completely around a thigh of the wearer of the hip orthosis on a same side of the sagittal plane as the first waist component; and
at least one connection component connected to a first anchor mount, the first anchor mount component affixed to the first waist component, wherein the at least one connection component is also connected to a second anchor mount, the second anchor mount affixed to the thigh component, and wherein the at least one connection component is connected to a third anchor mount, the third anchor mount affixed to the second waist component;
wherein the at least one connection component comprises a first element and a second element, wherein the first element of the at least one connection component is flexible and configured to transfer a force or forces between the first waist component and the thigh component, and wherein the second element of the at least one connection component is flexible and configured to transfer a force or forces between the second element and the second waist component; and
wherein a magnitude(s) and/or a direction(s) of the force or forces are configured to be adjustable by the wearer of the hip orthosis while the wearer is wearing the hip orthosis.

14) The hip orthosis of claim 13, wherein the force or forces partially or completely pull a leg of the wearer of the hip orthosis into adduction or adduces the leg of the wearer towards a midline of a body of the wearer of the hip orthosis.

15) A hip orthosis for providing a distraction force or forces comprising:
a leg portion for attaching to a leg of a wearer of the hip orthosis,
a waist portion for attaching to a waist of the wearer of the hip orthosis; and
at least one tensioning or compression apparatus comprising an adjustment mechanism and one or more cables directly or indirectly connecting the leg portion to the waist portion;
wherein the adjustment mechanism is configured to adjust and/or control a force or forces across and/or around one or more axes of rotation of a wearer's hip joint; and
wherein a direction of the force or forces results in a distraction force or forces applied to all or a part of a femur of the wearer of the hip orthosis relative to an acetabulum of the wearer of the orthosis, in at least one direction.

16) The hip orthosis of claim 15, wherein the leg portion and the waist portion are connected substantially continuously by the at least one tensioning or compression apparatus, the one or more cables, or a flexible garment portion.

17) The hip orthosis of claim 16, wherein the flexible garment portion comprises shorts, a sleeve, a compression garment, a pelvic garment, a textile panel, an elastic fabric, an inelastic fabric, or combinations thereof.

18) The hip orthosis of claim 15, wherein the at least one tensioning or compression apparatus comprises a strap, webbing, banding, ribbon, lace, cord, cable, rope, chain, or combinations thereof.

19) The hip orthosis of claim 15, wherein the at least one tensioning or compression apparatus comprises an inelastic strap.

20) The hip orthosis of claim 15, wherein the leg portion, the waist portion, or both, comprise one or more rigid or semi-rigid load-distribution members.

21) The hip orthosis of claim 16, wherein the one or more rigid or semi-rigid load-distribution members are coupled to or embedded within the flexible garment portion.

22) The hip orthosis of claim 15, wherein the adjustment mechanism comprises a buckle, cam buckle, ladder lock, ratchet, dial, reel, lever, pull tab, hook-and-loop fastener, or combinations thereof.

23) The hip orthosis of claim 15, further comprising one or more anchor regions on the thigh portion, the waist portion, or both, wherein one or more locations of the at least one tensioning or compression apparatus relative to the one or more anchor regions determines a direction of the distraction force or forces.

24) The hip orthosis of claim 23, wherein at least one of the one or more anchor regions comprises a reinforced textile region, sewn pathway, loop, guide, channel, ring, buckle, slot, hook-and-loop attachment region, rigid insert, semi-rigid insert, or combinations thereof.

25) The hip orthosis of claim 15, wherein the waist portion and/or the leg portion further comprise a waist belt, pelvic belt, torso sleeve, abdominal portion, back portion, hip portion, or combinations thereof.

26) The hip orthosis of claim 15, wherein the distraction force or forces are configured to reduce contact force between a femoral head and an acetabulum of the wearer of the hip orthosis.

27) A distraction orthosis for healing cartilage in a joint comprising, a first component, a second component, and a distraction mechanism, wherein the first component is disposed on a first side of the joint, wherein the second component is disposed on the second side of the joint, wherein the first component and the second component are connected by the distraction mechanism, and wherein the distraction mechanism produces a force or forces that pull, push, twist, or combinations thereof, the first component away from the second component, thereby generating a space between bones or cartilage on either side of the joint.

28) The distraction orthosis of claim 27, wherein the orthosis is worn by a wearer of the distraction orthosis more than two hours in a same day.

29) The distraction orthosis of claim 27, further comprising a safety feature wherein the safety feature limits the distraction force or forces and/or a distraction distance or distances.

30) The distraction orthosis of claim 27, further comprising one or more sensors and/or one or more indicators that sense or provide data on an amount of the distraction force or forces and/or a distraction distance or distances.

31) The distraction orthosis of claim 30, further comprising a microcontroller that logs, displays, transmits, and/or processes, the data sensed or provided by the sensors and/or indicators.

32) The distraction orthosis of claim 30, wherein the microcontroller adjusts servo(s), motor(s), actuator(s), or combinations thereof, to generate a predetermined distraction force or forces and/or predetermined distraction distance or distances.

33) A hip orthosis comprising a torso component, a thigh component, and at least one connection component, a first anchor attached to the torso component, a second anchor attached to the thigh component, wherein the torso component is secured partially or completely around a wearer's torso, wherein the thigh component is secured partially or completely around a wearer's thigh, wherein the at least one connection component is connected to both the first anchor and the second anchor, wherein the at least one connection component transfers a tensile force or forces between the torso component and the thigh component, which is configured to augment a contraction force of the gluteus medius muscle of the wearer of the hip orthosis, and wherein a magnitude and/or direction of the tensile force or forces can adjusted by wearer whilst the hip orthosis is being worn.

34) The hip orthosis of claim 33, wherein the tensile force or forces transferred by the at least one connection component partly or completely pull or force a leg of the wearer of the hip orthosis into abduction.

35) The hip orthosis of claim 34, wherein the abduction force or forces are transferred during a stance phase and a swing phase of a gait cycle of the wearer of the hip orthosis.

36) The hip orthosis of claim 33, wherein the at least on connection component comprises a cabling system.

37) The hip orthosis of claim 33, wherein the magnitude and/or direction of the force or forces transferred by the at least one connection component are altered by at least one adjustment component, and wherein the at least one adjustment component comprises a dial upon which a portion of the at least one connection component is wound.

38) The hip orthosis of claim 33, wherein the magnitude of the force or forces transferred by the at least one connection component are altered by at least one adjustment component, and wherein the direction of the force or forces transferred by the at least one connection component are altered by the same or a second adjustment component.

39) The hip orthosis of claim 36, wherein the cabling system does not comprise a closed loop.

40) The hip orthosis of claim 33, wherein the waist component includes a shoulder strap that prevents the waist component from migrating downwards when the force or forces are transferred between the waist component and the thigh component by the at least one connection component.

41) The hip orthosis of claim 33, further comprising a first connection component that transfers a tensile force or forces between the torso component and the thigh component, and a second connection component that limits a range or ranges of motion between the torso component and the thigh component.

42) The hip orthosis of claim 33, wherein the torso component and the thigh component are worn on a same side of a sagittal plane of the wearer of the orthosis.

43) The hip orthosis of claim 33, wherein the at least one connection component comprises an energy storage element in-line with a flexible element.

44) A hip orthosis comprising:
a first waist component;
a second waist component;
a thigh component; and
at least one connection component comprising at least three anchors, wherein the first waist component is secured partially or completely around a waist of a wearer of the hip orthosis, wherein the second waist component is secured partially or completely around the wearer's waist across a sagittal plane from the first waist component, the thigh component is secured partially or completely around a thigh of the wearer on a same side of the sagittal plane as the first waist component;
wherein a first anchor of the at least one connection component is affixed to the first waist component, a second anchor of the at least one connection component is affixed to the thigh component, and a third anchor of the at least one connection component is affixed to the second waist component; and
wherein a first element of the at least one connection component is flexible and the first element is configured to transfer a force or forces between the first waist component and the thigh component, wherein a second element of the at least one connection component is flexible and the second element is configured to transfer a force or forces between the first element and the second waist component, wherein a magnitude and/or a direction of the force or forces is configured to be adjustable by the wearer whilst the hip orthosis is being worn by the wearer.

45) The hip orthosis of claim 44, wherein the force or forces pull a leg of the wearer into adduction.
